(19) United States
(12) Patent Application Publication
(10) Pub. No.: US 2002/0182207 A1
(43) Pub. Date: Dec. 5, 2002

(54) Methods and compositions for modifying biologically active target molecules

(76) Inventor: Martin T. Martin, Rockville, MD (US)

Correspondence Address: Kramer Levin Naftalis & Frankel LLP, 919 THIRD AVENUE, NEW YORK, NY, 10022, US

(21) Appl. No.: 10/039,471

(22) Filed: Oct. 19, 2001

Related U.S. Application Data
(60) Provisional application No. 60/242,125, filed on Oct. 20, 2000.

(51) Int. Cl. A61K 39/395; A61K 31/545; A61K 38/43

(52) U.S. Cl. 424/130.1; 424/94.1; 514/44; 514/200

(57) ABSTRACT

The present invention provides a method of modifying a target substance by contacting the target substance with a catalyst that catalyzes the modification of the target substance. In a preferred embodiment, the method comprises labeling a target substance by contacting the target substance with a label and a catalyst that catalyzes the attachment of the label to the target substance. Preferably, the catalyst catalyzes selectively the reaction between a specific target molecule and a specific label. The attachment of labels may be used to inactivate a biologically active target substance or otherwise modulate its activity. Alternatively, the method may be used to label the target molecule with a detectable label suitable for the sensitive detection of the target substance.

### CROSS-REFERENCE TO RELATED APPLICATIONS

This application is claiming benefit from provisional application No. 60/242,125, filed on Oct. 20, 2000.

Reference is made to the following patent applications and issued patents:

U.S. Pat. Nos. 4,888,281, 5,037,750, 5,156,965, 5,855,885, 6,066,448, 5,731,147, 5,935,779; U.S. patent application Ser. Nos. 08/007,684, filed Jan. 22, 1993, 07/761,868, filed Sep. 3, 1991, 09/076,325, filed May 11, 1998, 08/447,515 filed May 23, 1995, 08/447,506, filed May 23, 1995, 08/485,324, filed Jun. 7, 1995, and 08/235,437, filed Apr. 29, 1994. The disclosures of each of these references are incorporated herein by reference.

### FIELD OF THE INVENTION

The present invention relates to methods and compositions for modifying target molecules. The instant method consists of contacting the target molecule with a catalyst that is capable of modifying the target molecule. The invention includes the use of such methods and compositions for labeling or modulating the activity of biological molecules, or for targeting biological molecules for degradation and/or clearance from the body. Preferably, the catalyst is a catalytic antibody isolated from a library of antibodies by phage display, in vivo selection, and/or high throughput screening.

Documents cited in this application relate to the state-of-the-art to which this invention pertains. The disclosures of each of these references are incorporated herein by reference.

### BACKGROUND OF THE INVENTION

Monoclonal antibodies (mAbs) are rapidly growing in importance as therapeutic drugs. Established techniques for the generation of monoclonal antibodies have enabled the isolation of monoclonal antibodies directed to a broad range of disease-specific proteins. In vivo, the binding of these mAbs to disease-specific proteins provides therapeutic benefits through a variety of mechanisms, including: (i) deactivation of the protein, and (ii) targeting the protein for degradation or clearance by the body. The tight binding affinities and exquisite specificities of monoclonal antibodies provide for specific targeting of disease-specific proteins with minimal side-reactions.

There is, however, a significant problem associated with the use of monoclonal antibodies. Each antibody is capable of binding at most one target protein per binding site. Therefore, the required dosages of antibody are often very high, as are the costs of the therapy. One approach to eliminating these problems is the use of catalytic antibodies (also known as abzymes). Catalytic antibodies, like traditional mAbs, have tremendous versatility as a class of reagents (catalytic antibodies have been made that catalyze a wide variety of types of reactions involving diverse types of substrates), while individual clones can have excellent specificity in the substrates they accept and the reactions they catalyze.

Catalytic antibodies, however, have additional advantages when compared to traditional mAbs. First, one catalytic antibody can potentially catalyze the destruction of a large number of target proteins. Further, the action of a catalytic antibody is a potentially irreversible chemical reaction, whereas traditional mAbs participate in an often strong but inherently reversible binding interaction. It has been suggested that catalytic antibodies having specific proteolytic activity against target proteins could have useful therapeutic value. However, there has been only limited exploration of other types of catalytic reactions that could also be useful for destroying or deactivating target proteins.

### OBJECTS OF THE INVENTION

It is therefore an object of the present invention to provide a method of modifying a biologically active target molecule consisting of contacting the target molecule with a catalyst capable of chemically modifying the target molecule. In a preferred embodiment, the catalyst is a catalytic antibody, the target molecule is a protein or peptide associated with a disease condition, and the catalytic antibody is capable of labeling the target molecule and thereby deactivating the target molecule. In a further preferred embodiment, the catalytic antibody is capable of labeling the target molecule by acylation with at least one β-lactam antibiotic, and the target molecule is selected from TNFα, IL-4, IL-6, VEGFr2, and CD3ε.

Additionally, it is an object of the invention to provide a catalyst capable of chemically modifying a biologically active target molecule, such as TNFα, IL-4, IL-6, VEGFr2, and CD3ε. In a preferred embodiment, the catalyst is a catalytic antibody and the chemical modification deactivates the target molecule.

It is a further object of the invention to provide compositions and methods for treating a disease condition associated with a target molecule by administering an effective amount of a catalyst capable of modifying a biologically active target molecule. Preferably, the catalyst is a catalytic antibody that labels and thereby deactivates the target molecule, and the compositions and methods are selective for a target molecule such as TNFα, IL-4, IL-6, VEGFr2, and CD3ε.

It is a further object of the invention to provide compositions and methods for labeling a target molecule by contacting the target molecule with a label and a catalyst capable of catalyzing the attachment of the label to the target molecule. In a preferred embodiment, the catalyst specifically catalyzes the labeling of the target molecule of interest and the label is a detectable label suitable for the sensitive detection of the target molecule. In another preferred embodiment, the attachment of the label modulates the biological activity of the target molecule or targets it for degradation or clearance.

### SUMMARY OF THE INVENTION

Using the methods of the present invention, one can circumvent the problems associated with traditional monoclonal antibodies by developing therapeutic methods to modify target molecules. These methods include ways of targeting molecules using catalytic reactions that have not been previously explored by those using catalytic antibodies.

The present invention provides a method of modifying a target substance by contacting the target substance with a catalyst that catalyzes the modification of the target substance. In a preferred embodiment, the method comprises labeling a target substance by contacting the target substance with a label and a catalyst that catalyzes the attachment of the label to the target substance. Preferably, the catalyst catalyzes selectively the reaction between a specific target molecule and a specific label. The attachment of labels may be used to inactivate a biologically active target substance or otherwise modulate its activity. Alternatively, the method may be used to label the target molecule with a detectable label suitable for the sensitive detection of the target substance.

The present invention provides novel catalysts, preferably catalytic antibodies, which can modify target molecules associated with disease conditions. The catalysts of the invention modify target molecules by a variety of methods, including labeling the target with a detectable moiety, linking one target molecule to another, modulating the activity of the target molecule, or targeting the molecule for degradation and/or clearance. Thus, the present invention provides a novel and effective means of selectively targeting such molecules in vivo.

The present invention provides a method of modifying a biologically active target molecule consisting of contacting the target molecule with a catalyst capable of chemically modifying the target molecule. In a preferred embodiment, the catalyst is a catalytic antibody, the target molecule is a protein or peptide associated with a disease condition, and the catalytic antibody deactivates the target molecule by acylation, glycosylation, or esterification. In a further preferred embodiment, the catalytic antibody is capable of modifying the target molecule by acylation with at least one β-lactam antibiotic and the target molecule is selected from TNFα:, IL-4, IL-6, VEGFr2, and CD3ε.

The present invention also provides a method of eliciting and isolating novel catalytic antibodies. Preferably, proteins or nucleic acids with the desired catalytic activity are identified by directed evolution. Such methods include i) the screening of large libraries of proteins, nucleic acids or organisms for members having the desired catalytic activity and ii) the selective growth or amplification of proteins, nucleic acids or organisms under conditions that favor individuals having the desired catalytic activity. In an especially preferred embodiment, the catalyst is a catalytic antibody and the catalytic antibody is isolated from a library of antibodies or fragments thereof by phage display, in vivo selection, and/or high throughput screening.

Additionally, the present invention provides catalysts, such as catalytic antibodies, that are capable of chemically modifying and thereby deactivating a biologically active target molecule, such as TNFα:, IL-4, IL-6, VEGFr2, and CD3ε. The invention also provides labels that can be attached to target molecules in the presence of such catalysts. The invention further provides compositions and kits that contain one or more members of the group selected from: i) a target molecules, ii) a catalyst capable of labeling said target molecule and iii) a label capable of being attached to target molecule in the presence of the catalyst.

Finally, the invention provides compositions and methods for treating a disease condition by administering an effective amount of a catalyst, preferably a catalytic antibody, capable of modifying a biologically active target molecule. Preferably, the compositions and methods comprise administering a catalytic antibody directed to TNFα, IL-4, IL-6, VEGFr2, and CD3ε, wherein the catalytic antibody deactivates the target molecule.

### BRIEF DESCRIPTION OF THE FIGURES

FIG. 1 shows the reaction that is believed to occur during the spontaneous labeling of proteins with β-lactam antibiotics.

FIG. 2 shows the chemical structures of ampicillin, cefoxitin, and cefotaxime.

FIG. 3 shows an expression vector for producing recombinant antibody in CHO cells.

### DETAILED DESCRIPTION OF THE PREFERRED EMBODIMENTS

The invention includes methods for modifying a target substance. The term “modify” or “modification” includes any method of chemically altering the structure of a target substance via a bimolecular reaction with a reagent other than water. The term “modification” includes (a) labeling the target molecule with a label; (b) linking one target molecule to another target molecule; (c) up or down modulating the biological activity of the target molecule; and (d) deactivating and/or degrading the target molecule and/or targeting the molecule for clearance from the body or transport into specific organs of the body. Modification includes reactions such as acylation, esterification, phosphorylation, sulfonation, nucleophilic substitution, electrophilic substitution, oxidation, reduction, glycosylation, transamidation, and the formation of Schiff bases from carbonyls and amines. Preferred embodiments of the invention include methods of labeling a target substance with a label. The term “labeling” is used broadly to describe the attachment of chemical groups or moieties other than water to a target substance; the term label refers to the chemical group or moiety that is attached. It is understood that there may be some changes in the structure and composition of the label and target during the attachment as long the product comprises some portion of both the target and the label. For example, the attachment of an aldehyde-containing label to an amine-containing target will lead to a target-label conjugate linked via a Schiff's base and the loss of a molecule of water.

The Target Substance

The target substance is preferably a biological substance such as a protein, nucleic acid, carbohydrate, cell, subcellular particle, prion, virus, phospholipid, etc. and/or a substance with biological activity such as a receptor, ligand, hormone, gene, gene message, enzyme, cytokine, etc. The term biological substance is meant to include synthetic substances that are designed to mimic, at least in part, the behavior or structure of naturally occurring substances: e.g., nucleic acid or protein analogs having unnatural monomeric units (i.e., unnatural nucleotides or amino acids) or linkages (e.g., peptide nucleic acids or peptides having all secondary amide linkages). Preferably, the target molecule has a biological activity that is relevant to a disease state so that inactivation or modulation of that biological activity has therapeutic relevance. Substances that are targeted by therapeutically relevant non-catalytic antibodies are especially attractive targets for the catalytic reactions of the invention due to the aforementioned higher efficiency of catalytic therapeutic agents. Some examples of biological target substances associated with a disease condition include: TNFα, IL-4, IL-6, VEGFr2, CD3ε, IL-1, TGF-β, gp120, IgE CD45, CD33, EGF receptor, CD20, CD40, HER2/neu, HER2 receptor, TNFα receptor, VEGF, 2B1, IgE, ICAM-1, CD6, CD18, hCG, CD25, IL-2, IL-2 receptor, CD58, α4-integrin, β2 integrin, A4b7 integrin, FcγR1, TAG-72, hepatitis B virus, DNA Histone H1 complex, gpIIbIIIA, ICAM-3, CD4, CD11, CD18, CD28, CD2, CD80, CD48, HLA-Dr10, CBL, respiratory syncytial virus, CD52, IL-8, and CA125.

The Catalyst

The catalyst is a substance that catalyzes the modification and, preferably, the labeling of the target substance. Catalysts of biological origin such as enzymes, catalytic antibodies, and catalytic nucleic acids, are preferred because these classes of substances include members displaying a wide range of catalytic activities, because these catalytic activities are often quite substrate specific, and because of the known biological and biochemical methods for generating diverse populations of these substances which can be screened for activity.

Catalytic antibodies are especially preferred catalysts. Like monoclonal antibodies (mAbs), catalytic antibodies offer exquisite specificity for disease-associated proteins. However, unlike mAbs, each catalytic antibody can react with multiple target molecules, thereby permitting dramatic enhancements in therapeutic efficacy. Lower doses are required to achieve the same result. This would lead to a reduced incidence of harmful side effects, which are typically dose-dependent. Thus, the therapeutic index of a catalytic antibody is expected to be higher than that for mAbs.

In therapeutic applications, it is advantageous if the catalyst has a slow clearance rate from the patient's blood so that the doses can be kept low and infrequent. Antibodies are known to have a long residence time in blood. It is particularly advantageous if the antibodies are human in origin. The use of human antibodies minimizes problems due to immune reactions and maximizes the lifetime of the antibodies in blood. Alternatively, an antibody of non-human origin can be “humanized” by forming and expressing a genetic construct that codes for an antibody having the variable regions of the non-human antibody but the constant regions of a human antibody. Another approach for increasing the lifetime of non-human antibodies and reducing problems from immune reactions is to modify the antibody with molecules such as oligo-ethylene glycols that protect the antibody from enzymatic proteolysis and recognition by the patient's immune system.

The Label

The label is any chemical group or moiety that can be linked to the target substance. In one embodiment of the invention, the label is a detectable label that is suitable for the sensitive detection of the target substance. Examples of detectable labels include luminescent labels (e.g., fluorescent, phosphorescent, chemiluminescent, bioluminescent and electrochemiluminescent labels), radioactive labels, enzymes, particles, magnetic substances, electroactive species and the like. Alternatively, a detectable label may signal its presence by participating in specific binding reaction. Examples of such labels include haptens, antibodies, biotin, streptavidin, his-tag, nitrilotriacetic acid, glutathione S-transferase, glutathione and the like. In an alternate embodiment of the invention, the label need not be detectable but instead functions to modulate the biological activity of a target substance. The attachment of one or more labels to a target substance may interfere with the catalytic activity of a catalytic active site in the substance (e.g., when the target substance is an enzyme) or may prevent the recognition of the target substance by a binding partner of the target substance. Alternatively, the label may be a signaling moiety that targets the target substance for degradation (e.g., ubiquitin) or that targets the target substance for transport, e.g., to a specific tissue or to a specific region of a cell (see, e.g., Lindgren et al., Trends in Pharmacol. Sci., 2000, 21, 99). For therapeutic applications, the free label should be relatively non-toxic so that it can be maintained in a patients blood at high concentrations, preferably greater than 1 uM. Some examples include sugars, β-lactam antibiotics and isoniazid.

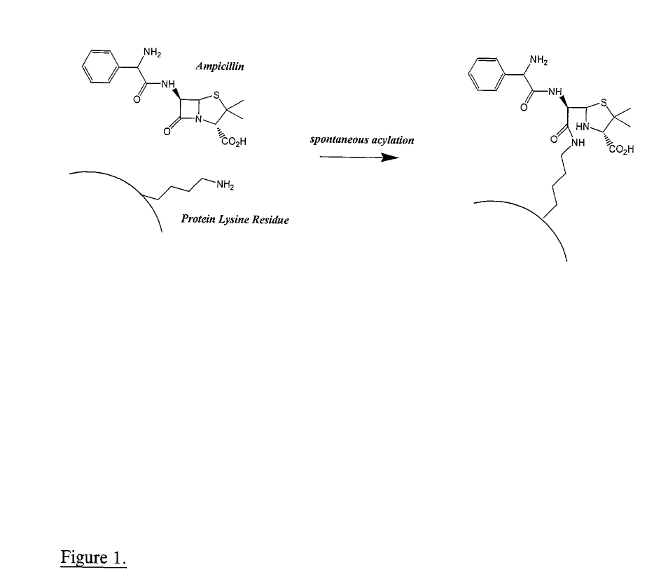

Preferably, the target substance and the label have some low but detectable rate of reaction in the absence of catalyst. Some examples of such reactions include: i) the reaction of nucleophiles such as amines, hydroxyls or thiols with activated acyl species such as esters, thioesters, amides, anhydrides, acyl halides, acyl phosphates, isothiocyanates, cyanates, carbamates, carbonates, amides (especially high energy amides such as β-lactams) and the like; ii) the reaction of nucleophiles such as amines, hydroxyls or thiols with activated phosphorous or sulfur compounds such as halophosphates, phosphoramidates, sulfonyl halides and the like; iii) the reaction of amines with aldehydes (or alternatively, hemiacetals or acetals) or ketones (or alternatively, hemiketals or ketals) to form Schiff bases; iv) electrophilic substitution (e.g., iodination of tyrosines in proteins); and v) nucleophilic substitution (e.g., the reaction of amines, thiols and hydroxyls with alkyl halides). There are well-known examples of uncatalyzed chemical labeling reactions that occur in the bloodstream. For example, the aldehyde groups in circulating carbohydrates react with amines in the environment. In a specific example of this type of reaction, glucose spontaneously reacts with protein lysine residues to form covalent “advanced glycation end products” (AGE). The resulting glycated proteins cause diabetic complications. Another example is the acylation of protein lysine residues by β-lactam antibiotics (see FIG. 1). Individuals that have penicillin allergies are actually not allergic to the penicillins themselves, but rather to penicillin-protein (usually albumin) conjugates.

The fact that an uncatalyzed reaction can occur, albeit at a slow rate, in the absence of the catalyst places a lower burden on the catalyst and makes it more likely that a catalyst can be found without extensive screening. For example, in some cases it is only necessary that the catalyst bind to both the target substance and the label so as to hold them in close proximity and, therefore, increase the effective concentrations of the reacting species.

Generation of Catalytic Antibodies by Immunization

Usually catalytic antibodies are elicited by immunizing mice with a transition state analog (TSA) of the desired reaction. A transition state of a reaction is a fleeting high-energy intermediate that appears during the course of the reaction, usually for no more than 10^{-—}seconds. Enzyme theory states that enzymes are catalytic because their active sites are complementary to the transition state. Antibodies that bind to a TSA should also be complementary to the actual transition state, and thus they should be catalytic.

A second strategy for creating catalytic antibodies is the so-called “bait-and-switch” approach. This method involves designing and preparing an immunogen that carries a charge opposite to that desired in the antibody binding site. Antibodies that bind to this immunogen are likely to have a charge that is complementary to that of the binding site. This charged antibody participates in general acid-base reactions during catalysis.

A third strategy is to use the catalytic antibody combining site as an “entropy trap”. In the case of bimolecular reactions, if the antibody binds both substrates simultaneously in a productive orientation the reaction is greatly accelerated by a proximity effect. Studies with catalytic antibodies have shown that the substrate's effective molarity can exceed 100 M. This method does not require synthesizing a transition state analog of the reaction, but rather the generation of antibodies that bind to a compound that resembles the two substrates or product in the target bimolecular reaction. Although this strategy has been successful, it is not often used because most catalytic antibody reactions are not bimolecular.

Generation of Catalysts by Screening Libraries for Binding Activity

The TSA, bait and switch, and entropy trap strategies work by selecting for catalytic antibodies that have some binding affinity for haptens resembling the starting materials, intermediates or products along a reaction pathway. In the bait and switch method the hapten is modified in a way designed to select for a catalytic residue in the binding pocket of the catalytic antibody. A variety of methods now exist that allow one to carry out these types of selection processes based on binding affinity without requiring the use of live animals and without limiting the catalysts to catalytic antibodies. Peptide display technologies such as phage display, yeast display, bacterial display, viral display, ribosome display, RNA-protein fusions, etc. allow for the simultaneous screening of large number of peptides and the selective enrichment and amplification of peptides that participate in a desired binding interaction; see, e.g., the following references hereby incorporated by reference: U.S. Pat. No. 5,403,484, U.S. Pat. No. 5,223,409, WO98/31700, WO99/36569, Hanes et al. (Proc. Natl. Acad. Sci., 1997, 94, 4937). The peptide libraries may be random peptide libraries, libraries of antibodies or antibody fragments, or enzyme libraries. Through the use of error-prone PCR one can produce displayed libraries having randomly mutated forms of a particular protein, antibody or enzyme. Techniques similar to the peptide display technologies also exist for selecting nucleic acid sequences with specific binding properties (e.g., the SELEX method as described in U.S. Pat. No. 5,475,096). In a preferred embodiment, the catalyst is an antibody that is isolated from a phage library.

It is advantageous to begin with a large library of potential catalysts and to first reduce the size of the library by enrichment binding to the desired reaction product (as described below in the case of antibody libraries on phage) so as to simply further screening and increase the likelihood of finding molecules with the desired catalytic activity. There are two reasons for pre-selecting antibodies on the basis of product binding. Due to the nature of phage display technology, only a portion of phage in the library displays a functional scFv. Pre-selection will enrich those that display a scFv. The second reason is that it is desirable to use a subset of potential compounds that are more likely to contain the desired catalyst. Thus, by pre-selecting a subset of antibodies that, at least weakly, bind to the reaction product (antibiotic-target adduct), the chances of finding a catalyst are much greater. In a preferred embodiment, the antibody library is reduced from greater than 10⁹ (most preferably greater than 10¹²) clones to less than or around 5×10⁴ (most preferably less than or equal to 10⁴).

To pre-select antibodies that have some affinity for the product, the uncatalyzed reaction may be used to chemically prepare the reaction product, e.g., a β-lactam-target protein conjugate. The target protein conjugate is generated in a purified or partially purified form (i.e., 50 to 90% homogeneity). The human antibody phage library is then “panned” against the conjugate. In panning, phage antibodies are incubated in a plastic tube containing surface-coated antigen. A wash step is employed to strip off non-specifically bound phage, after which bound phage are removed by elution with a high pH buffer. Typically, multiple rounds of panning are carried out. In the screening of potential catalysts, it is preferable to retain as much diversity as possible and to retain both strong and weak binding antibodies. Thus, the selection process is limited to a single round of panning and the number of wash steps of the tube after antigen selection and prior to elution should also be minimized. Preferably, the number of clones eluted from the tube after a single panning step is approximately 1-5×10⁴.

Phage display is a technique in which large collections of filamentous bacteriophage particles (often exceeding 10¹⁰ unique particles) are used as tools to discover unique peptides or proteins. All of the phage in a library are physically identical except that each particle displays 1-5 copies of a unique protein or peptide on its surface. By applying a specific selection method (e.g., binding or catalysis) to the bulk phage library, phage displayed proteins with the desired properties can be isolated. The beauty of this technique is that the isolated phage particles physically contain the gene that encodes the displayed protein. Hence, the production of the peptide or protein can be easily scaled up, and the peptide or protein may be readily purified and characterized. Methods for the phage display of antibodies are described in U.S. Pat. No. 5,855,885.

Many types of peptides and proteins have been used in phage libraries, including small peptides (Chiniros-Rojas et al., 1998), enzymes (Demartis et al., 1999), and antibodies (Winter et al., 1994). Phage libraries have been used to isolate candidate therapeutic antibodies (Huls et al., 1999; Mao et al., 1999) and catalytic antibodies (Arkin & Wells, 1998; Fujii et al., 1998).

The high molecular weight disulfide-linked tetrameric structure of natural IgG molecules is difficult to express in E. coli . Therefore the preferred form for antibody phage display is the single chain Fv or scFv. The 25 kDa scFv molecules consist of only the variable heavy and light chain regions of antibodies, connected by a short peptide linker, which fold to form a functional antibody binding site. If desired, scFv molecules can be easily re-engineered to Fab, full-sized IgG, or other molecular forms. It is also advantageous to include in the nucleic acid sequence encoding the scFv fragment a sequence that codes for a purification tag such as FLAG, (his)₆, glutathione S-transferase, maltose binding protein, etc.; the purification tag allows for the straightforward purification of the scFv fragment using an affinity column or solid phase specific for the purification TAG sequence (e.g., a column comprising, respectively, anti-FLAG antibody, nickel-NTA, glutathione, amylose, etc.).

Because the process of assembling and displaying the immune repertoire on phage is performed ex vivo, it is likely that the potential number of antibody fragments with catalytic activity is much larger then would be found in vivo. This is theoretically possible because the pairing of the variable heavy and light chain domains that comprise the binding pocket is completely random and combinatorial, and not restricted in any way by normal immunological regulation.

In a preferred embodiment, the phage are panned to reduce the library size and then E. coli is infected with the resulting phage and this sub-library is used in the directed evolution work. It should be noted that the same sub-library of antibodies can be used in the HTS experiments described below.

Directed Evolution of Pre-Selected Antibodies

Directed evolution is the generation of a large number of mutants of a chosen gene, followed by the selection of mutant genes which express a protein with desired characteristics, i.e., an in vivo selection method to discover novel desirable proteins. Directed evolution differs from natural evolution in two key respects. First, genetic variation is introduced by the experimenter rather than by nature. This allows a rapid method of producing greater number of diverse mutants than would occur naturally. Second, in directed evolution, novel proteins are obtained by either natural selection (e.g., using auxotrophic bacteria) or by individual or bulk screening of the generated novel proteins (e.g., high throughput screening or phage display).

In one example, an antibody library is screened for the desired activity. Preferably, the library is pre-enriched for molecules that bind to the desired reaction product (as described above). The diversity of such a library can be further increased while maintaining a high probability of hits by introducing a limited amount of random mutations into the antibody library. The library of antibodies can be screened by HTS (e.g., by running a biochemical assay for the desired activity on each individual clone or antibody) or by natural selection (e.g., by genetically introducing the library into cells and applying a selective pressure that limits the growth of cells that do not produce an antibody with the correct activity). Typically, when using natural selection to screen for a catalyst that catalyzes the labeling of a target molecule, the target and/or the label are capable of providing negative selective pressure on the organism when they are not linked, but the labeled target is incapable of exerting this negative selective pressure. For example, if the target is a hormone with a negative regulatory activity in the host cell, the screening will select for cells expressing catalysts that label the target in a way that destroy this biological activity. Similarly, the label may be a molecule that negatively regulates or kills the cells being used to express the library, e.g., an antibiotic.

Such techniques can also be applied to the selection of enzymes with a desired catalytic activity. In this case, it is advantageous to start with an enzyme with an activity similar to the desired activity and use directed evolution to screen for mutants of that enzyme with the desired activity. For example, mutants of a β-lactamase are screened for catalysts that catalyze the labeling of proteins with β-lactam antibiotics (i.e., the water nucleophile in the hydrolysis of a β-lactam is replaced with the ε-amino group of a lysine on the target molecule). In a second example, a variety of relatively non-specific trans-amidases are known that can attach amine containing labels to proteins via linkage to the side chains of asparagine or glutamine residues. In this case, mutants are screened for transamidase mutants that retain this activity but that are specific for certain target proteins and/or labels. It is known, for example, that many aryl hydrazides, such as the anti tuberculosis drug isoniazid, are covalently attached to serum proteins in vivo due to the action of serum transglutaminases (see, e.g., Lorand et al., Biochemistry, 1972, 11, 434). Mutants of these or other transamidases can, therefore be screened for catalysts that efficiently and/or specifically label a specific target protein with isoniazid.

Methods for the directed evolution of desired target molecules are disclosed in U.S. Pat. Nos. 5,837,500, 5,571,698, 5,223,409, 5,096,815, and 5,258,289, the disclosures of which are incorporated herein by reference.

Ladner et al., U.S. Pat. No. 5,096,815, describes a method of developing novel DNA-binding proteins and polypeptides by an iterative process of mutation, expression, selection, and amplification. Briefly, Ladner et al. uses a variegated population of DNA molecules, each encoding one of a large number of potential target-binding proteins, to transform a cell culture. The cells of the culture are engineered with binding marker genes so that, under selective conditions, the cell thrives only if the expressed target-binding protein binds to the target subsequence, thereby preventing expression of these binding marker genes. The mutant cells are directed to express the potential target-binding proteins and the selection conditions are applied. Cells expressing proteins binding successfully to the target are thus identified by in vivo selection. The process is repeated until a protein or polypeptide with the desired binding characteristics is obtained.

Preferably, the mutant cells are provided with a selectable gene coding on expression for a product deleterious to the survival or growth of the cell, operably linked to a promoter regulating the expression of the gene. The promoter is modified to include the desired target subsequence in a position where it will not interfere with expression of the selectable gene unless a protein binds to that target subsequence. Each mutant cell is also provided with a gene encoding on expression a potential DNA-binding protein, operably linked to a promoter that is preferably regulated by a chemical inducer. When this gene is expressed the potential DNA-binding protein has the chance to bind to the target and thereby protect the cell from the selective conditions under which the product of the binding marker gene would otherwise harm the cell.

Davis et al., U.S. Pat. No. 5,258,289, provides an alternative selection method, which is specifically tailored to the screening and selection of catalytic antibodies capable of cleaving a specified peptide sequence. Briefly, a target peptide is chosen, together with a phage gene that encodes a gene product necessary for the production of a phage. The phage gene is modified by introducing the target peptide coding sequence into the gene such that the resulting gene product inhibits production of infectious phage, and cleavage of the target peptide results in an active gene product that allows production of infectious phage. The modified phage is introduced into a host with a library of rearranged immunoglobulin genes in a cloning vector, which library is capable of expressing immunoglobulin genes in the cloning vector, under suitable expression conditions. The host cells are grown under conditions in which the immunoglobulin genes are expressed in the host cells, and the presence of antibodies capable of cleaving the target peptide is identified on the basis of phage production.

β-Lactam antibiotics have been used previously as the selection pressure against E. coli in directed evolution studies. In those cases, the bacterial enzyme β-lactamase was the protein being subjected to directed evolution. β-Lactamase hydrolytically destroys β-lactam antibiotics such as the penicillins and is usually responsible for bacterial resistance to antibiotics. This enzyme is particularly amenable to directed evolution since mutant enzymes with improved catalytic activity will give the host organism greater antibiotic resistance. Thus, those bacteria with efficient mutant enzymes will survive an antibiotic challenge.

Directed evolution may be used to identify catalysts that modify target proteins, e.g., by acylation with β-lactam antibiotics. β-Lactam antibiotics (including the penicillins and cephalosporins) are toxic to bacteria when the four-membered heterocyclic β-lactam ring is intact, but are completely non-toxic after the ring is opened by hydrolysis or acylation. Briefly, antibodies in an antibody library are individually expressed in and secreted by E. coli. The target protein can either be added to the growth medium or co-expressed with the antibody. A toxic level of β-lactam antibiotic is added to the E. coli colonies. Any organism that secretes a catalytic antibody that can catalyze the acylation of a target protein with antibiotic will survive because the process of acylation (ring opening) inactivates the antibiotic.

Although the reaction of ampicillin with proteins was facile and gave the desired result (biological inactivation), there are two problems with using penicillins such as ampicillin as the chemically modifying reagent: 1) the scFv expression plasmid in a common type of phage antibody library encodes a penicillin-hydrolyzing β-lactamase, and 2) penicillin allergies will ultimately limit the use of catalytic antibody therapies based on penicillin substrates.

Therefore, efforts were made to identify β-lactam antibiotics that are non-allergenic (cephalosporins rather than penicillins) and that are not recognized and hydrolyzed by E. coli β-lactamase. Four candidate cephalosporins were identified: cefacler, cephalothin, cefoxitin, and cefotaxime. All are FDA-approved antibiotics and fairly inexpensive from a common commercial source (Sigma Chem. Co.). In addition, they are not as likely to be as allergenic as the penicillins. The incidence of allergic reactions to cephalosporins is very low and any reactions that may occur are likely to be mild (e.g., rash, urticaria).

First, the ability of these candidate cephalosporins to kill the relevant β-lactamase-producing E. coli (TG1 phage antibody E. coli) was tested. The results showed that ampicillin, cefaclor, and cephalothin have little or no antibiotic effect at the indicated concentrations. This is presumably due to β-lactamase-catalyzed inactivation. However, both cefoxitin and cefotaxime showed excellent antibiotic effects. Presumably, neither is inactivated by E. coli β-lactamase. By analyzing the relative toxicities of these antibiotics it was found that E. coli should preferably be challenged with 30-50 μM cefoxitin and 0.30-0.60 μM cefotaxime. More preferably, E. coli should be challenged with approximately 40-45 μM cefoxitin and 0.45-0.50 μM cefotaxime.

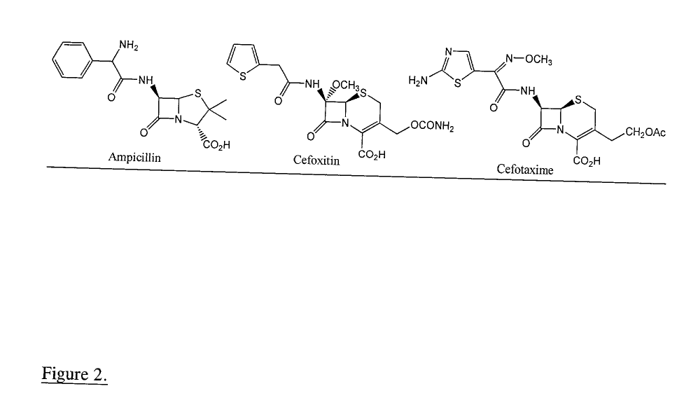

The structures of ampicillin, cefoxitin, and cefotaxime are shown in FIG. 2.

High-Throughput Screening

Libraries of potential catalysts may be screened via the testing of individual members of the library (or alternatively, small groups of members). A variety of techniques and instrumentation are available for the rapid conduct of large numbers of individual biological or biochemical tests. These techniques and instruments fall under the general heading of high throughput screening. Typically, HTS tests are carried out in multi-well plates that have a large number of isolated wells that can be used to carry out individual tests (a variety of standardized plates for HTS have been made including plates with 96, 384, and 1536 wells). A variety of instrumentation exists for carrying out measurements of the properties of individual wells, such as optical absorbance, fluorescence, phosphorescence, chemiluminescence, electrochemiluminescence, radioactivity, etc. The use of these plates, instrumentation for conducting measurements on the plates, and robotics for dispensing fluids to and from the plates, allows an HTS screener to conduct large numbers of assays in parallel (e.g.,>10,000 individual measurements in a day). Some alternative non-plate based approaches also exist such as parallel capillary electrophoresis or mass spectrometry.

There are several approaches for assaying for catalytic activity. For example, a potential catalyst may be combined with the target molecule and label and the consumption of the target and label and/or the production of labeled-product can be measured. Suitable techniques for directly measuring target, label and labeled-target include mass spectrometry and chromatographic techniques such as capillary electrophoresis. Alternatively, the reaction may be followed via a change in a spectroscopic property such as optical absorbance, fluorescence, chemiluminescence, electrochemiluminescence, etc. Another suitable approach is to measure changes in the amount of free target, free label or labeled-target via specific binding assays such as immunoassays. For example, the target, label and potential catalyst are incubated and a sandwich immunoassay is carried out for the target; significant labeling will reduce the ability of antibodies to recognize target and will result in a reduction of the immunoassay signal. In a second example, a sandwich immunoassay is carried out using a first antibody that is specific for the target molecule and a second antibody that is specific for the label; catalytic activity can be detected in this case via the formation of a sandwich complex comprising the first antibody, the second antibody and the labeled target. In a third example, a sandwich immunoassay is carried out using two antibodies directed against the label (e.g., in a solid phase immunoassay, one is labeled and the other is immobilized); in this case, catalysis that results in multiple labeling of the target will result in a signal via the formation of a sandwich complex comprising the two antibodies bound top a multiply labeled target molecule. In a fourth example, catalytic labeling is measured via the ability of the label to disrupt the interaction of the target with a receptor specific for the target. Binding assays for detecting the products of catalytic labeling reactions may be carried using any one of a variety of binding assay detection methods and instrumentation known in the art (see, e.g., The Inmunoassay Handbook 2^{nd} Edition, David Wild, Ed., Groves Dictionaries Inc.: New York, 2000). The assays may be in homogeneous or heterogeneous formats. In a preferred embodiment, electrochemiluminescence detection is used.

Preferably, the library that is screened by HTS is pre-enriched in catalysts with some binding affinity to the desired reaction product. However, the rapid advancements in HTS technology now allow for the rapid testing of extremely large libraries (>10⁶ ) so depending on the diversity of the original library, in many cases pre-enrichment will not be required. The combination of phage display with HTS is unprecedented and it enables the detection and isolation of rare, remarkably efficient catalytic antibodies.

Target Molecules

Various protein targets have been identified and tested as suitable for the method of the present invention, including, but not limited to TNFα, IL-4, IL-6, VEGFr2, CD3ε, IL-1, TGF-β, gp120, CD45, CD33, EGFr, CD20, CD40, HER2/neu, HER2 receptor, TNFα receptor, VEGF, 2B1, IgE, ICAM-1, CD6, CD18, hCG, CD25, IL-2, CD58, α4-integrin, gpIIbIIIA, ICAM-3, CD4, CD11, CD18, CD28, CD2, CD80, CD48, respiratory syncytial virus, CD52, IL-8, or CA125 antigen. In a preferred embodiment, the biological molecule is TNFα, IL-4, IL-6, VEGFr2, and CD3ε. The skilled artisan will readily appreciate that the instant method is not restricted to use with these specific target proteins. These proteins were initially chosen because inactivation of each was very likely to have a significant therapeutic effect. All of the targets have the following characteristics: known three-dimensional structure; surface lysine residues believed to be critical for biological function; numerous indications in the literature that inactivation of the target will result in some therapeutic effect in an important disease state; non-catalytic antibody therapies exist that show a beneficial effect that could be enhanced using a catalytic version; and published cloning procedure and nucleotide sequence.

Several of these target proteins are discussed in more detail below.

i. TNFα

TNFα is a 17 kDa cytokine that is involved in many diverse biological processes. TNFα is a mediator in a number of pathological states including inflammation, septic shock, cachexia, cancer, Crohn's disease, parasitic infections, allograft rejection, and heart disease. It exerts its effects by binding to its receptors TNFα-R1 (55 kDa) and TNFα-R2 (75 kDa), both of which are present on virtually all cell membranes.

Anti-TNFα binding proteins are effective therapeutic agents in rheumatoid arthritis and Crohn's disease. The FDA has approved two protein drugs that bind TNFα: Enbrel and Centocor. Enbrel (Etanercept, Immunex and Wyeth-Ayerst) is a recombinant human TNFα p75 receptor-Fc fusion protein (TNFR:Fc). Enbrel was approved to treat rheumatoid arthritis in November 1998. Centocor is a chimeric anti-TNFα antibody (hifliximab, Remicade, cA2) that is approved for use in the treatment of Crohn's disease. Because TNFα is involved in so many different disease processes, it is likely that TNFα blockers will eventually become useful elsewhere as well. For example, the Centocor antibody is in clinical trials as an arthritis treatment. In addition to the use of binding proteins to target TNFα, there are various other strategies for inhibiting TNFα function. However, other approaches do not specifically target TNFα (for example, protein synthesis inhibition) and hence may have side effects.

TNFα is an attractive target for use in the method of the present invention because its inhibition has been shown to be therapeutically effective (FDA-approved in rheumatoid arthritis and Crohn's disease). Further, the structure and function of TNFα are well known. Human TNFα was cloned many years ago and an essential surface lysine has been identified through crystallographic and structure/function studies. Finally, reducing sugars and penicillins inactivate TNFα. These reactions are uncatalyzed and suggest that biocatalysts can be developed to accelerate the reaction rates and make the reactions TNFα-specific.

TNFα is a trimer consisting of three identical 17 kDa subunits. Its structure and receptor binding interface are well characterized. Lysine 71 is essential for receptor binding. Without wishing to be bound by any theory, it is likely that chemical modification of this residue caused the inactivation by reducing sugars and β-lactams in the experiments described above.

ii. VEGF Receptor 2

Vascular endothelial growth factor (VEGF) is an endothelial cell-specific mitogen and angiogenesis inducer produced by a variety of tumor cell lines. VEGF is critical to normal angiogenesis and pathological processes such as tumor growth, ocular neovascularization, and rheumatoid arthritis. In humans, there are two known VEGF receptors, VEGFr1 (flt1) and VEGFr2 (KDR). Only VEGFr2 mediates endothelial cell proliferation and angiogenesis.

There are a number of human cancer clinical trials ongoing in which angiogenesis factors are targeted. Solid tumors must be well vascularized to obtain nutrition to grow. Anti-angiogenesis strategies seek to inhibit neovascularization by blocking the action of tumor-secreted angiogenesis factors. One approach (Genentech, Inc., South San Francisco) uses an antibody to block the effects of VEGF. The antibody binds to VEGF rather than to the receptor. Genentech's anti-VEGF antibody is in Phase III clinical trials for treatment of metastatic renal cell cancer. The VEGF receptor, and VEGFr2 in particular, is likely to be a superior target to VEGF itself in treating cancer. First, VEGFr2 is present in higher concentrations than VEGF. Moreover, VEGFr2 is always present, whereas levels of VEGF may vary. In addition, VEGF, but not its receptors, are subject to alternative exon splicing, resulting in multiple protein isoforms. Although the drug is not yet in clinical trials, work is underway to develop cancer therapeutics using an anti-VEGFr2 antibody (ImClone Systems, Inc.). Thus far, the antibody appears to be effective at inhibiting tumor growth.

Historically, antibody therapies of solid tumors have not been very successful, primarily because the large size of antibody molecules (150 kDa) makes it difficult for them to penetrate the microvasculature of the tumor to kill cells deep within the mass. However, targeting angiogenesis is one way to circumvent this issue. If angiogenesis is halted at the surface of the tumor mass, nutrients will not reach any of the tumor cells, surface or internal, and the tumor will die. Encouraging results from clinical trials using an anti-VEGF antibody (noted above) lend credence to this hypothesis.

VEGF receptors form a subfamily within the platelet-derived growth factor (PDGF) receptor class. All VEGF receptors consist of seven immunoglobulin (Ig) homology domains, a transmembrane sequence, and an intracellular split kinase domain. VEGFr2 is 200 kDa, and the binding site for VEGF has been mapped to the 97 amino acid second domain of the VEGFr2 Ig domain. The deduced amino acid sequence of VEGFr2 is known and the molecule has been cloned and expressed. The second Ig domain has five lysine residues. It is likely that at least one of these lysines is critical for VEGF binding.

iii. Interleukin 4 (IL-4)

IL-4 is a 20 kDa glycoprotein produced mainly by the T helper lymphocyte type 2 (T_{H}2) cell population. IL-4 (as well as IL-5 and IL-13) recruits and activates IgE-producing B cells, eosinophils, and mast cells. IL-4 plays a pathological role during allergic inflammations associated with allergic asthma, rhinitis, conjunctivitits, and dermatitis.

Evidence suggests that an anti-IL-4 immunotherapy can be effective in treating asthma. The soluble IL-4 receptor is currently being studied in human clinical trials as an asthma treatment (Immunex Inc., Seattle, Wash.). It would be advantageous to develop catalytic antibodies that inhibit the biological function of IL-4, which can be used as anti-asthmatics. These catalytic antibodies could also useful in other IL-4-dependent diseases, such as graft-versus-host disease and allergies.

Human IL-4 is a short-chain 4-helix bundle cytokine. High-resolution 3-dimensional structures of both IL-4 and its receptor are known. The receptor contacts have been identified. IL-4 binds to its receptor with a subnanomolar range dissociation constant. This tight binding is largely a result of mixed charge pairs between known surface amino acids. In particular, solvent-exposed lysines 77 and 84 on helix C appear to be vital to receptor binding. Lysine 12 is also a modifiable essential residue. The labeling of these lysine residues by a bulky cephalosporin should severely reduce or eliminate the binding affinity between IL-4 and its receptor. Finally, IL-4 was first cloned over a decade ago and its nucleotide sequence is known.

iv. Interleukin 6 (IL-6)

IL-6 is a multifunctional cytokine that plays roles in immune responses, inflammation, hematopoiesis, and in the nervous and endocrine systems. IL-6 also induces B cells to differentiate into antibody-producing plasma cells, contributes to T-cell growth and differentiation, and is a hematopoietic growth factor. Among pathological functions, IL-6 is a growth factor for myeloma and plasmacytoma cells, renal cell carcinoma, and Kaposi's sarcoma. It is also contributes to arthritic inflammation.

IL-6 is a good target for catalytic antibody therapy in multiple myeloma (MM), rheumatoid arthritis (RA), Castleman's Disease, AIDS, and other diseases. In particular, catalytic antibody therapy would be attractive in multiple myeloma because of the high IL-6 concentrations often observed. It has been postulated that conventional (non-catalytic) anti-IL-6 immunotherapies would be ineffective in advanced MM because the IL-6 concentrations are 25 times higher than antibody concentrations in high dose immunotherapy. This situation is ideal for a catalytic antibody because even a weakly catalytic antibody can easily catalyze 25 turnovers before being cleared from circulation. Clinical trials are underway with non-catalytic anti-IL-6 antibodies for the treatment of large-cell lymphoma, MM and renal cell carcinoma, rheumatoid arthritis, and Castleman's disease.

IL-6 was discovered in the early 1980's and cloned in 1986. It is a glycoprotein of 21-30 KDa, depending on the cellular source and preparation method. The heterogeneity is due to variations in posttranslational modification. A high resolution X-ray structure of this four helix bundle protein has been recently published, which has helped to clarify many previous structure-function studies. Taken together, a number of structural, mutagenesis, and functional studies have been carried out to delineate the amino acids in IL-6 required for binding to its receptor. The IL-6 receptor is also well characterized. These studies indicate that lysine 28 is critical for binding of IL-6 to its receptor, and that lysines 55 and 128 are essential for biological activity.

V. CD3ε

About 20,000 patients receive organ transplants in the U.S. each year. Rejection is the most common cause of transplant failure, occurring in greater than 80% of solid organ transplant recipients. In order to prevent or treat this potentially fatal immune system response, transplant patients must take immunosuppressive medications. Depending on the patient's condition, different therapies are mandated. Small molecule immunosuppressive drugs such as cyclosporin or tacrolimus, mycophenolate mofetil or azathioprine, and prednisone are taking prophylactically post-surgically to reduce the likelihood of rejection. In cases of acute rejection, where the patient's T-cell lymphocytes attack the graft, higher doses of immunosuppressants, corticosteroids or monoclonal antibody therapies. Current mAbs for acute transplant rejection are muromonab-CD3 (OKT3, Orthoclone OKT3) by Ortho/J&J, which targets the CD3ε protein; basiliximab (Simulect) by Novartis; and daclizumab (Zenapax) by Roche. The latter two antibodies are anti-IL2 receptor agents and are also administered before and after surgery to reduce the likelihood of rejection in the first year. OKT3 is administered prophylactically only in cases where the likelihood of rejection is high (Wilde and Goa, 1996).

Antigen recognition by T-cells involves a complex between heterodimeric T-cell receptor (TCR) and the CD3 complex. CD3 consists of at least three different proteins, γ, δ, and ε. CD3ε is present on all T-cells and is absolutely required for T-cell activation (Elgart, 1996; Imboden, 1997).

Many antibodies that bind to CD3ε disrupt T-cell function, resulting in an immunosuppressive effect (Pescovitz, 1999; Bostrom & Ringden, 1995; Halloran & Prommool, 1998; Smith & Bluestone, 1997; Alegre et al., 1997). CD3ε is by far the most antigenic CD3 subunit, as most anti-CD3 antibodies bind to CD3ε (Tunnacliffe et al., 1989; Transy et al., 1989; Portoles et al, 1999). Because of their immunosuppressive activity, anti- CD3ε antibodies are effective in prevention and treatment of rejection of transplanted organs and bone marrow. Most anti-T-cell antibody treatments can deplete greater than 99% of circulating T-cells (Bostrom & Ringden, 1995). Anti- CD3ε antibodies may also be useful in the treatment of T-cell tumors (Ma et al., 1997).

The first FDA-approved therapeutic monoclonal antibody was an anti- CD3ε antibody called OKT3 (Burk & Matuszewski, 1997; Pescovitz, 1999; Halloran & Prommool, 1998; Smith & Bluestone, 1997). OKT3, approved in 1980, is far from a perfect therapeutic agent because of severe side effects. Many of the adverse side effects arise as a consequence of OKT3 being a mouse antibody. In addition, because OKT3 has a Fc region, problems can occur due to cell-cell crosslinking. Ortho/J&J sells OKT3 (muromonab-CD3, Orthoclone OKT3) for use in transplantation.

Recently, improved anti- CD3ε antibodies have been reported that were prepared by humanizing mouse antibodies and inactivating the Fc region (Klingbeil & Hsu, 1999; Pescovitz, 1999; Smith & Bluestone, 1997). Protein Design Labs, Inc. has an engineered anti-CD3ε antibody that has done well in clinical trials (HuM291, SMART anti-CD3) (Klingbeil & Hsu, 1999). Most antibodies directed against CD3ε are naked antibodies, which act by blockading the biological activity of CD3. An alternative approach uses an anti-CD3 antibody to direct diphtheria toxin to T-cells (Ma et al., 1997). In addition, it should be noted that a number of other immunosuppressive antibodies (and other biological materials) are under development. Some would be redundant with anti-CD3 therapy (Halloran & Prommool, 1998; Alegre et al., 1997). For example, anti-IL2 receptor treatments are approved for transplantation—daclizumab (Zenapax, Roche) and basiliximab (Simulect, Novartis).

As discussed above, CD3 consists of at least three different protein subunits. The epsilon subunit is a 20-kDa non-glycosylated transmembrane protein. It consists of an amino-terminal 104 amino acid extracellular segment, a 26 amino acid hydrophobic transmembrane segment, and a 79 amino acid intracellular carboxyl terminus (see Elgart, 1996; Gold et al., 1996; Borroto et al., 1998).

CD3ε has a number of physical features that make it attractive as a target. It has been cloned, it is small (104 amino acid extracellular segment), and it is not glycosylated or otherwise post-translationally modified. Its folded structure is well understood. Five of the 104 extracellular amino acids are lysine residues—targets for catalyzed chemical modification (Elgert, 1996; Gold et al., 1986; Borroto et al., 1998). Anti-CD3ε antibodies are well-established therapeutic agents that have been in clinical use for 20 years.

The extracellular 104 amino acid segment of CD3ε is cloned, expressed, and purified. The cloned protein segment is used as a catalytic antibody substrate in HTS and in directed evolution.

A clone that contains the coding sequence for the T-cell surface protein CD3ε is available from ATCC (#1397503). With the clone, the complete insert sequence is determined by DNA sequencing and compared to the previously published sequence of CD3ε cDNA (Gold et al., 1986). The protein consists of a total of 185 amino acids, with a structure shown in the diagram below (Huppa and Ploegh, 1997).

For bacterial expression of CD3ε, the DNA sequence corresponding to the extracellular domain is amplified by PCR using specific primers. During this process, specific cloning sites will be added to the 5′ and 3′ ends of the amplified product, to allow subsequent cloning into an E. coli protein expression vector. A number of such vectors are available commercially, for example pET, which can be used to achieve high-level, secreted expression of the cloned CD3ε protein. Secreted expression of the protein is an important aspect of the directed evolution approach for isolating catalytic antibodies that specifically modify CD3ε. In addition, the secreted CD3ε protein is purified to homogeneity from E. coli cell paste for use in preparing the β-lactam-CD3ε protein conjugate. This conjugate is required for panning the phage antibody display library prior to isolating specific antibody catalysts using either HTS or directed evolution. Purification is facilitated by expressing the CD3ε with a HIS₆ tail, a common component of most E. coli expression vectors, which allows for protein isolation in a single step using immobilized metal affinity chromatography (IMAC).

Two (reduced) cysteine residues (97 and 100) are in the extracellular domain near the transmembrane segment, which begins with valine 105 (see diagram above). Because thiols could cause technical problems due to oligomerization or misfolding, these residues are removed from the expressed protein, using either an appropriate restriction enzyme to eliminate the cysteines by gene truncation or by performing site-directed mutagenesis to change them to alanine residues. For reference, the denoted extracellular loop is formed by a disulfide bridge involving cysteines 27 and 76. The exposed targeted lysine residues are numbers 15, 42, 51, 63, and 78. (Gold et al., 1986; Borroto et al., 1998)

A number of anti-CD3ε monoclonal antibodies are commercially available that are used in immunoassays to detect the chemical modification of CD3ε. The antibodies are labeled with an activated NHS ester derivative of the ECL compound Ru (bpy)₃²⁺ (IGEN Intl., Inc.). A sandwich assay specific for unlabeled CD3ε should use an anti-CD3ε antibody that binds to native CD3ε but not to the β-lactam-CD3ε conjugate. It is also important that the antibody has been shown in the literature to disrupt T-cell function as a result of CD3ε binding (i.e., both the antibiotic and the antibody localize to a shared physiologically essential epitope). A number of suitable antibodies are commercially available.

Assay development requires the preparation of the antibiotic-CD3ε complex. As described above, the spontaneous reaction can be carried out between the antibiotic(s) and cloned and expressed CD3ε for 2-3 days, resulting in modified protein. Mild neutral, aqueous conditions can be used. The conjugate can easily be purified by dialysis and column chromatography.

Labeled anti-CD3ε antibodies are then screened for binding to the antibiotic-CD3ε conjugate. The conjugate is immobilized according to standard ECL methods (e.g., by using a second anti-CD3ε antibody that is immobilized on streptavidin-coated magnetic beads) and antibody binding is detected by electrochemiluminescence (ECL) detection. Antibodies that bind to recombinant CD3ε but not to the antibiotic- CD3ε conjugate are usable in immunoassays of catalytic activity.

Some sources of appropriate anti-human CD3ε monoclonal antibodies are:

R&D Systems, Inc., Minneapolis, Minn. (Catalog # MAB100)

ATTC, Manassas, Va. (antibody OKT3 (Cat. # CRL-8001) and antibody BC3 (Cat. # HB-10166))

BD Pharmingen, San Diego, Calif. (clones 1D4.1, 8D3.1, SP34)

Caltag Laboratories, Burlingame, Calif. (clone MEM57)

Accurate Chemical & Scientific Co., Westbury, N.Y. (clone MEM57)

Research Diagnostics, Inc., Flanders, N.J. (clone CLBT3-4E).

A human phage antibody repertoire display library is panned against the antibiotic-CD3ε conjugate. The resulting subset of ˜10⁴ antibodies is then subjected to HTS and directed evolution.

The antibiotic-target protein conjugate for panning is prepared by prolonged incubation of the two reaction components, CD3ε and antibiotic. The rate and yield of the uncatalyzed reaction is optimized by varying the reaction conditions (time, temperature, pH, etc.). The rate of the reaction is monitored by following the loss of CD3ε antibody binding. The immunoassay described above is used to monitor the reaction progress. Appropriate controls are used to ensure that the loss of CD3ε is not due to an artifact such as proteolysis or denaturation.

Once antibiotic-CD3ε conjugate has been formed, the reaction mixture is dialyzed to remove unreacted antibiotic and exchange buffer. The conjugate is adsorbed onto a plastic tube. Next, the entire human scFv phage library (˜10¹² antibodies) is added to the tube for panning. Wash conditions are varied to determine a suitable amount of washing to adequately reduce background phage binding without compromising the diversity of specifically selected phage. Bound phage are eluted using pH shock and the resulting eluate is infected into E. coli and plated on selective media to obtain isolated colonies.

Approximately 10,000 antibodies are screened for catalytic activity. Phage antibodies are expressed in E. coli, and the supernatants from individual clones are screened by immunoassay in an IGEN M-SERIES ECL instrument.

In directed evolution experiments, E. coli colonies representing the panned scFv library are challenged with toxic doses of either Cefoxitin or Cefotaxime. If E. coli secretes a scFv that can catalyze the conjugation of the antibiotic to secreted CD3ε, then the antibiotic is inactivated and that colony will survive. Colonies that do not secrete abzymes do not inactivate the antibiotic and are not selected. The concentrations of antibiotic used is twice the IC₅₀, or 44 μM Cefoxitin and 0.50 μM Cefotaxime (see above).

The simplest way to include the target protein and the antibiotic is to add them to the agar media used for the selection. This is reasonable for the antibiotic, but the amount of target protein required for selection on Cefoxitin is on the order of 44 μM, which in the case of CD3ε requires as much as 500 milligrams to perform the selection using 1000 mL of media. The cost to perform the selection in this manner is prohibitive. Selection on Cefotaxime requires 10-fold less protein, which is more feasible. A second, more attractive approach is to co-express the target protein in the same bacterial cell as the scFv. The addition of a signal sequence directs the expression of the protein to the bacterial periplasm, the same location as the expressed scFv protein. The two major advantages of this approach are: 1) lower cost compared to adding the target protein to the media 2) a more favorable environment for catalysis as a consequence of concentrating the three components of the reaction in the bacterial periplasmic space.

Co-expression of the scFv and target protein in the same bacterial cell is achieved by the following method: following selection, eluted phage (about 10 ⁴ pfu) are infected into an E. coli strain that harbors a plasmid that expresses and secretes CD3ε. The infected cells are then pelleted by centrifugation, resuspended in a suitable volume of media and plated on agar medium containing the appropriate concentration of Cefoxitin or Cefotaxime. After a suitable incubation, any colonies that appear are isolated, regrown, and stored in glycerol at −80° C. Expressed scFv from each colony is then be purified on a large-scale and tested for catalytic activity.

Catalytic antibodies (scFv) discovered by either HTS or directed evolution are produced in E. coli, then purified by standard methods. Catalytic activity is verified on pure antibody. Characteristic kinetic parameters (k_{cat}, Kₘ and k_{cat}/Kₘ) are determined. Finally, screening and characterization of any inhibitors, including substrate and product inhibition is carried out. The most attractive catalytic antibody (in terms of efficiency and stability) is advanced to the next phase of the project, molecular engineering.

Catalytic activity of discovered abzymes is determined by two methods. In an indirect method, antibody-catalyzed conjugation reactions are quenched at suitable times using 10 mM NaOH. Sodium hydroxide terminates the catalyzed reaction and hydrolyzes intact (unreacted) antibiotic. Upon hydrolysis, the Uv/Vis absorbance of free antibiotic changes substantially (λ for Cefoxitin is 800 M⁻¹cm⁻¹ (290 nm) and 544 M⁻¹cm⁻¹ (330 nm) for Cefotaxime), allowing residual (unreacted) antibiotic to be measured. The second method is ECL-based immunoassay of the intact target molecule. This method directly indicates target inactivation, but is somewhat more time-consuming than the indirect method. Both methods are used in a complementary manner.

Although the scFv antibodies are ideal for cloning, expression, and phage display, its small size relative to Fab fragments or whole IgG may diminish their therapeutic efficacy due to its short serum half-life. Therefore, conversion of the scFv abzymes to an IgG is desirable for developing an effective therapeutic agent. A number of vector systems have been described in the literature for producing recombinant antibodies in vitro. The advantage of this vector is that the antibody heavy and light chain genes are on a single plasmid as opposed to two separate plasmids, thereby simplifying the cell transformation and clone selection process. The steps to convert an scFv to whole IgG are relatively straightforward and involve re-cloning the VH and VL domains of the scFv into the appropriate sites of the IgG expression vector, followed by transfection into Chinese hamster ovary (CHO) cells. Once IgG producing clones are identified, they are grown to a larger scale in stir flasks and the IgG purified from culture supernatants using Protein G chromatography.

Because the catalytic antibodies have been reengineered at this point from a scFv to a whole IgG, its kinetic and stability characteristics my change. The kinetic parameters should not change significantly, but whole IgG stability should be considerably greater than the scFv from which it was derived. Characterization experiments that were initially done to characterize with the scFv are repeated with the IgG molecule.

Thus, the method of the present invention may be used to develop therapeutic catalytic antibodies to treat a variety of autoimmune and inflammatory diseases and cancer, including one or more of the following conditions:

1. Rheumatoid arthritis, an autoimmune disorder resulting in severe inflammation of the joints, afflicts 2.7 million patients in the U.S. and greater than 5 million worldwide. It is estimated that approximately 270,000 rheumatoid arthritis patients in the U.S. are candidates for anti-TNFα therapy.

2. Crohn's disease is a serious inflammatory condition of the gastrointestinal tract. There are approximately 400,000 Crohn's patients in the U.S., about 250,000 of whom have moderate to severe disease. All of these patients are candidates for anti-TNFα therapy.

3. Asthma afflicts about 17 million people in the U.S. and about 20 million in Europe and Japan. More than half of these people are candidates for long-term control therapy, such as the anti-IL-4 therapy described above.

4. Multiple myeloma is the second most common hematological malignancy in the U.S. According to the American Cancer Society, there were approximately 13,800 new cases of multiple myeloma diagnosed in the U.S. in 1998 and 11,300 deaths from the disease. The only curative therapy is a combination of chemotherapy and stem cell transplantation. Chemotherapy alone can prolong life but most current treatments focuses on palliation. All multiple myeloma patients would be potential candidates for a therapeutic antibody having greater efficacy and fewer side effects than chemotherapy.

5. Colorectal cancer will be the first target disease for anti-VEGFr2 catalytic antibody. The incidence of colorectal cancer worldwide is 876,000, with 130,200 new cases diagnosed each year in the U.S. About 56,300 people die of colorectal cancer each year in the U.S. Surgery can be curative in stages I and II of the disease. Advanced stage II and III patients receive adjuvant chemotherapy to prevent recurrence. Patients with metastatic disease receive chemotherapy to prolong survival. Based on the incidence of the different stages of colorectal cancer about 70% of colorectal cancer patients could be candidates for treatment with a therapeutic catalytic antibody, in either the adjuvant or metastatic disease settings.

6. Treatment of rejection of organ transplants using an anti-CD3E catalytic antibody. Of the 20,000 organ transplants performed per year, rejection is the most common cause of transplant failure, occurring in greater than 80% of solid organ transplant recipients.

Preferred Formulations and Routes of Administration

The catalysts of the present invention may be administered either alone or in combination with other active agents or compositions typically used in the treatment or prevention of the above-identified disease conditions. Such active agents or compositions include, but are not limited to steroids, non-steroidal anti-inflammatory drugs (NSAIDs), chemotherapeutics, analgesics, immunotherapeutics, antiviral agents, antifimgal agents, vaccines, immunosuppressants, hormones, cytokines, antibodies, antithrombotics, cardiovascular drugs, or fertility drugs. Also included are vaccines, oral tolerance drugs, vitamins and minerals.

Catalysts may be administered intravenously or in the form of a liquid or semi-aerosol via the intratracheal tube. Other viable routes of administration include topical, ocular, dermal, transdermal, anal, systemic, intramuscular, slow release, oral, vaginal, intraduodenal, intraperitoneal, and intracolonic. Such compositions can be administered to a subject or patient in need of such administration in dosages and by techniques well known to those skilled in the medical, nutritional or veterinary arts taking into consideration such factors as the age, sex, weight, and condition of the particular subject or patient, and the route of administration. The compositions of the present invention may also be administered in a controlled-release formulation. The compositions can be co-administered or sequentially administered with other active agents, again, taking into consideration such factors as the age, sex, weight, and condition of the particular subject or patient, and, the route of administration.

Examples of compositions of the invention include edible compositions for oral administration such as solid or liquid formulations, for instance, capsules, tablets, pills, and the like liquid preparations for orifice, e.g., oral, nasal, anal, vaginal etc., formulation such as suspensions, syrups or elixirs; and, preparations for parenteral, subcutaneous, intradermal, intramuscular or intravenous administration (e.g., injectable administration), such as sterile suspensions or emulsions.

In such compositions, the catalyst(s) may be in admixture with a suitable carrier, diluent, or excipient such as sterile water, physiological saline, glucose, DMSO, ethanol, or the like. The catalyst can be provided in lyophilized form for reconstituting, for instance, in isotonic aqueous, saline, glucose, or DMSO buffer.

Further, the invention also comprehends a kit wherein a catalyst is provided. The kit can include a separate container containing a suitable carrier, diluent or excipient. The kit can include an additional agent that reduces or alleviates the ill effects of the above-identified conditions for co- or sequential-administration. The additional agent(s) can be provided in separate container(s) or in admixture with the antibody. Additionally, the kit can include instructions for mixing or combining ingredients and/or administration.

### EXAMPLES

The invention will now be further described with reference to the following non-limiting examples.

Experiments were conducted to determine whether catalytic antibodies can chemically modify and thereby inactivate disease-associated proteins. First, the reaction of reducing sugars with the disease-associated protein, tumor necrosis factor alpha (TNFα) was examined. In this reaction, the reducing sugar aldehyde covalently reacts with protein lysine sidechains. Chemical modification was detected using an ECL-based binding assay that measures the binding of TNFα to its receptor (an assay of biological function). It was found that sugars gradually inactivate TNFα receptor binding over a 2-week incubation period, whereas the non-reducing sugar, sucrose, did not inactivate TNFα. Thus, chemical modification is a viable approach to inactivate proteins.

The uncatalyzed reaction between a β-lactam (ampicillin) and two proteins was also examined. Bovine serum albumin (BSA) and ampicillin were reacted overnight, and it was found that ampicillin groups were covalently attached to BSA. Ampicillin was also reacted with TNFα overnight. It was found that ampicillin caused a 16% loss of TNFα biological activity (according to the receptor binding assay). This reaction is much more facile than the reaction of TNFα with reducing sugars. This work shows that β-lactam antibiotics are effective modification labels in the inactivation of therapeutically-relevant target proteins.

Preferably, the catalyst is a catalytic antibody isolated from a library of antibodies or fragments thereof by one or more techniques selected from (i) phage display, (ii) in vivo selection, and (iii) high-throughout screening. Each of these methods are discussed in more detail below.

### Preselection of Antibodies by Phage Display

### Example 1

### Feasibility Studies

Experiments were conducted to determine whether catalytic antibodies can inactivate disease-associated proteins. First, the reaction of reducing sugars with the disease-associated protein, tumor necrosis factor alpha (TNFα) was examined. In this reaction, the reducing sugar aldehyde covalently reacts with protein lysine sidechains. Chemical modification was detected using an ECL-based immunoassay that measures the binding of TNFα to its receptor. The assay is a sandwich format that uses antibodies directed against TNF and its receptor. One antibody is labeled with biotin, the other with an ECL label (TAG NHS Ester, IGEN International). When the TNF-TNF receptor complex is present, a sandwich complex comprising both a biotin and an ECL label is formed. This sandwich complex is captured on streptavidin-coated magnetic beads and measured using ECL detection. Alternatively, the TNF receptor may be directly biotinylated and the TNF target molecule labeled directly with the ECL label (or visa versa); in this alternative approach, no antibodies are necessary. The results summarized below in Table 1 show that sugars gradually inactivate TNFα receptor binding over a 2-week incubation period. It was also found that the non-reducing sugar, sucrose, did not inactivate TNFα; the labeling by the reducing sugars, therefore, probably occurs via the formation of a Schiff's base with exposed lysines on the protein. Thus, chemical modification may be used to inactivate proteins.

TABLE 1
Loss of TNFα Receptor Binding Following Incubation with Various
Sugars

| Incubation | | | | |
|---|---|---|---|---|
| Time | Control (buffer) | + Glucose | + Galactose | + Fructose |
| None | 1.00 | 1.04 | 1.00 | 1.02 |
| | (arbitrary) | | | |
| 7 days | 1.00 | 0.82 | 0.89 | 0.79 |
| | (arbitrary) | | | |
| 14 days | 1.00 | 0.82 | 0.82 | 0.67 |
| | (arbitrary) | | | |

The uncatalyzed reaction between a β-lactam (ampicillin) and two proteins was also examined. Bovine serum albumin (BSA) and ampicillin were reacted overnight. Subsequent chemical analysis showed that ampicillin groups were covalently attached to BSA. Ampicillin was also reacted with TNFα overnight. It was found that ampicillin caused a 16% loss of TNFα biological activity according to the receptor binding assay. Thus, this reaction is much more facile than the reaction of TNFα with reducing sugars. This work shows that β-lactam antibiotics are effective modification labels in the catalytic antibody-catalyzed inactivation of therapeutically-relevant target proteins.

Next, the ability of the cephalosporins to kill the relevant β-lactamase-producing E. coli (TG1 phage antibody E. coli) was tested by growing the bacteria on agar plates containing the antibiotics. The results, summarized below in Table 2, show that ampicillin, cefaclor, and cephalothin have little or no antibiotic effect at the indicated concentrations. This is presumably due to β-lactamase-catalyzed inactivation. However, both cefoxitin and cefotaxime showed excellent antibiotic effects. Presumably, neither is inactivated by E. coli β-lactamase.

TABLE 2

| Antibiotic | 10 g/mL | 10 g/mL |
|---|---|---|
| Ampicillin | +++++ | +++++ |
| Cefaclor | +++++ | +++ |
| Cephalothin | +++++ | +++++ |
| Cefoxitin | +/− (30 colonies) | − |
| Cefotaxime | − | − |

A score of 5 + (++++++) indicates that growth was comparable to that of the ampicillin plate.

A second experiment was performed in which E. coli was grown on agar plates containing varying amounts of cefotaxime. The data in Table 3 show that the toxic level of cefotaxime is between 0.12 and 0.37 μg/mL.

TABLE 3

| Cefotaxime Concentration (μg/mL) | # of E. coli colonies |
|---|---|
| 0.00 | Lawn |
| 0.04 | 225 |
| 0.12 | 5 |
| 0.37 | 0 |
| 1.11 | 0 |

Thus, the concentrations of cefoxitin and cefotaxime that are toxic to β-lactamase-expressing E. coli are approximately 10 μg/mL (22 μM) and 0.12 μg/mL (0.25 μM), respectively. These low figures indicate that these antibiotics are unrecognized by β-lactamase. Preferably, the concentrations of these antibiotics used in directed evolution experiments are slightly higher, e.g., about two times higher, than these concentrations in order to ensure that no E. coli will survive without evolved mechanisms. Thus, E. coli is preferably challenged with 44 μM cefoxitin and 0.50 μM cefotaxime.

E. coli expresses antibody at approximately 90 nM. Thus, for a catalyst to hydrolyze one-half of the antibiotic challenge (which would bring it to the brink of survival), it would have to catalyze 244 turnovers (22 μM/0.09 μM) with cefoxitin or 14 turnovers (0.25 μM/0.09 μM) with cefotaxime. A requirement for multiple turnovers is advantageous to ensure that E. coli cannot survive by simple binding of the antibody to the antibiotic, but that catalysis is required to allow growth.

Further, before choosing either cefoxitin or cefotaxime as the labeling reactant, it was verified that their uncatalyzed reactivity rates were as facile as that of ampicillin. Model reactions were performed comparing the (NaOH) hydrolysis rates of cefoxitin, cefotaxime, and ampicillin. Hydrolysis and acylation are mechanistically identical. Their pseudo-first order hydrolysis rates as measured by UV spectrometry (1.0 mM antibiotic, 10.0 mM NaOH, 30.0° C.) were virtually the same:

| | |
|---|---|
| Ampicillin | 5.64 × 10⁻³ min⁻¹ |
| Cefoxitin | 4.38 × 10⁻³ min⁻¹ |
| Cefotaxime | 4.10 × 10⁻³ min⁻¹. |

### Example 2

### TNF-α

Phage Display

A human phage antibody repertoire display library is panned against the antibiotic-target protein conjugate. The resulting subset of ˜10⁴ antibodies is then subjected to HTS and directed evolution.

The antibiotic-target protein conjugate for panning is prepared by prolonged incubation of the two reaction components, TNFα and antibiotic. The rate and yield of the uncatalyzed reaction are optimized by varying the reaction conditions (time, temperature, pH, etc.), e.g., pH 5-9, 60 minutes-3 weeks, and 4-37° C. The rate of the reaction is monitored by following the loss of TNFα receptor binding. Appropriate controls, e.g., TNF alone or in combination with hydrolytically-inactivated antibiotic, are used to ensure that the loss of TNFα is not due to an artifact such as proteolysis or denaturation.

Once the antibiotic-TNFα conjugate is formed, the reaction mixture is dialyzed to remove unreacted antibiotic and exchange buffer. The conjugate is adsorbed onto a plastic tube. Next, the entire human scFv phage library (approximately 10¹² antibodies) is added to the tube for panning. Various wash conditions should be tested in order to determine a suitable amount of washing to adequately reduce background phage binding without compromising the diversity of specifically selected phage, preferably using a buffer of roughly neutral pH such as PBS. Bound phage is eluted using pH shock and the resulting eluate infected into E. coli and plated on selective media to obtain isolated colonies.

HTS

Approximately 10,000 antibodies are screened for catalytic activity during a two-week period. Phage antibodies are expressed in E. coli, and the supernatants are screened by immunoassay, for example by using ECL detection. The target protein is obtained as described in the phage display section above. In a typical assay protocol, the antibody, the target molecule (e.g., TNF) and the label (e.g., glucose or a beta-lactam) are combined and incubate (typically at 25-37 C for a period of 30 min. to 12 hours). The amount of TNF modification is determined using a binding assay that measures the amount of target protein capable of binding to the TNF receptor.

Directed Evolution

In directed evolution experiments, E. coli colonies representing the panned scfv library are challenged with toxic doses of either cefoxitin or cefotaxime. If E. coli secretes an scFv that can catalyze the conjugation of the antibiotic to secreted TNFα, then the antibiotic is inactivated and that colony will survive. Colonies that do not secrete catalytic antibodies do not inactivate the antibiotic and are not selected. The concentrations of antibiotic used is twice the IC₅₀, or 44 μM cefoxitin and 0.50 μM cefotaxime.

The target protein is co-expressed in the same bacterial cell as the scFv. The addition of a signal sequence directs the expression of the protein to the bacterial periplasm, the same location as the expressed scFv protein. The two major advantages of this approach are 1) lower cost compared to adding the target protein to the media 2) a more favorable environment for catalysis as a consequence of concentrating the three components of the reaction in the bacterial periplasmic space.

Co-expression of the scFv and target protein in the same bacterial cell is achieved by the following method:

After selection, eluted phage (about 10⁴ pfu) is infected into an E. coli strain that harbors a plasmid that expresses and secretes TNFα. The infected cells are pelleted by centrifugation, resuspended in a suitable volume of media and plated on agar medium containing the appropriate concentration of cefoxitin or cefotaxime. After a suitable incubation, typically overnight, any colonies that appear are isolated, regrown, and stored in glycerol at −80° C. Expressed scFv from each colony is then be purified on a large-scale, e.g., using a nickel-NTA column and tested for catalytic activity.

Characterization of Discovered Catalytic Antibodies

Catalytic antibodies (scFv) discovered by HTS and/or directed evolution are produced in E. coli, then purified by standard methods, e.g., by using a nickel-NTA column. Catalytic activity is verified on pure antibody. Characteristic kinetic parameters (k_{cat}, Kₘ and k_{cat}/Kₘ) are determined. Finally, screening and characterization of any inhibitors, including substrate and product inhibition are carried out.

Catalytic activity of discovered catalytic antibodies is determined by two methods. One method is indirect; catalytic antibody-catalyzed conjugation reactions is quenched at suitable times using 10 mM NaOH. Sodium hydroxide terminates the catalyzed reaction and hydrolyzes intact (unreacted) antibiotic. Upon hydrolysis, the UV/Vis absorbance of free antibiotic changes substantially (Δεfor cefoxitin is 800 M−¹cm⁻¹ (290 nm) and 544 M−¹cm⁻¹ (330 nm) for cefotaxime), allowing residual (unreacted) antibiotic to be measured. The second method is by specific binding assay for unmodified or modified target. This method directly indicates target inactivation, but is somewhat more time-consuming than the indirect method. Both methods are used in a complementary manner.

Re-engineering Catalytic Antibodies from scFv fragments to Whole IgG

Although the scFv antibodies are ideal for cloning, expression, and phage display, its small size relative to a whole IgG may diminish their therapeutic efficacy because of its short serum half-life. Therefore, conversion of the scFv catalytic antibodies to a whole IgG is preferable for developing an effective therapeutic agent. A number of vector systems have been described in the literature for producing recombinant antibodies in vitro. An example is shown below. The advantage of this vector is that the antibody heavy and light chain genes are on a single plasmid as opposed to two separate plasmids, thereby simplifying the cell transformation and clone selection process.

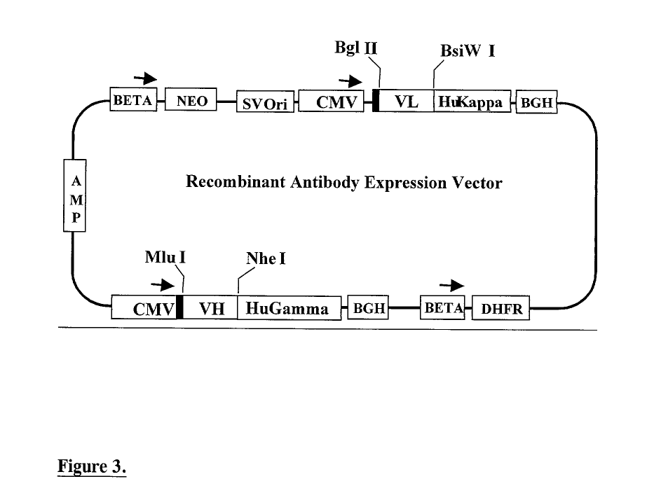

The scFv is converted to whole IgG by re-cloning the VH and VL domains of the scFv into the appropriate sites of the IgG expression vector (see FIG. 3) followed by transfection into Chinese hamster ovary (CHO) cells. Once IgG producing clones are identified, they can be grown to a larger scale in stir flasks and the IgG purified from culture supernatants using Protein G chromatography.

Re-Characterization of Catalytic Antibodies

Because the antibody has been reengineered from a scFv to a whole IgG, its kinetic and stability characteristics my change. While the kinetic parameters should not change significantly, whole IgG stability should be considerably greater than the scFv from which it was derived. Therefore, characterization experiments that were initially done to characterize with the scFv are repeated with the IgG molecule.

Biological Studies

General

The instant method enables one to isolate catalytic antibodies that have a beneficial effect in one or more non-human disease models. There are well-established animal models for all diseases described in this application. Moreover, in all cases, these animal models have been used to test (non-catalytic) immunotherapies directed toward the same target molecules.

Animal Models

There are a number of excellent animal models for RA. The most common model is cartilage-induced arthritis (CIA) in which collagen is injected into mice. Specifically, the DBA/1 mouse collagen type II animal model is used, in which both antibiotic (either cefoxitin or cefotaxime) and antibody are injected into the mouse using various reasonable dosing and timing regimens. The regimens depend on a number of predetermined factors, including the pharmacokinetics (blood level vs. time profile) and tolerance of the antibiotic and of the catalytic antibody. Any effects of the antibody are observable using the same parameters as were used in non-catalytic antibody animal trials, including footpad swelling, a marker of inflammation, clinical score, which measures the degree of inflammation and number of affected limbs, and joint destruction, assessed by histology.

An adjuvant-induced arthritis (AIA) rat model is also used to test the therapeutic effect of catalytic antibodies. This model is also well established and involves the induction of arthritis by a single subcutaneous injection of a mixture of killed mycobacteria and mineral oil (Freund's complete adjuvant). As with the CIA model in mice, any effects of the antibody are observable by footpad swelling, clinical score, and joint destruction.

### Example 3

### VEGFr2

Phage Display

Phage display is carried out as described in Example 2, with two exceptions. Catalytic activity is characterized by using a binding assay that measures the binding of VEGFr2 to VEGF, and the second IgG domain of VEGFr2 is cloned and expressed (for both directed evolution and HTS work). Alternatively, modification of VEGFr2 can be detected using a sandwich immunoassay using antibodies that are specific for unlabeled or slightly labeled protein.

Cloning of VEGFr2

Two lengths of VEGFr2 are cloned for different purposes. For directed evolution, the 97 amino acid second Ig domain is cloned and inserted into a plasmid to be constitutively expressed in E. coli. The entire seven Ig domain extracellular VEGFr2 segment is also cloned. The cloned, expressed, and purified extracellular domain is used in an immunoassay for VEGF binding to VEGFr2. The immunoassay is used to discover catalytic antibodies by HTS and in antibody characterization.

HTS

High throughput screening to discover catalytic antibodies capable of inactivating VEGFr2 is carried out as described above in Example 2, except the binding assay to detect catalysis is different. The binding assay tests the ability of VEGF to bind to the cloned extracellular VEGFr2. VEGF may be obtained commercially (Research Diagnostics, Flanders, N.J.) or cloned, expressed, and purified. Before the assay, VEGF is labeled with Ru(bpy)₃⁺², an electrochemiluminescent label, by using an NHS ester derivative (TAG NHS, IGEN International). The assay procedure involves incubating the catalytic antibodies with VEGFr2 and antibiotic. If, after incubation, Ru(bpy)₃⁺²-VEGF fails to bind to VEGFr2, it indicates that the antibody has catalyzed antibiotic-VEGFr2 conjugation. Loss of binding is detected using ECL-based HTS instrumentation.

Directed Evolution

Directed evolution to discover anti-VEGFr2 antibodies is carried out as described above in Example 2, except that, instead of TNFα, the second Ig domain of VEGFr2 is co-expressed with scFv molecules in E. coli.

Characterization of Discovered Catalytic Antibodies

Discovered antibodies are characterized as described above in Example 2, except the binding assay is specific for VEGFr2.

Re-Engineering Antibodies from scFv Fragments to Whole IgG

The scFv fragments are converted to whole IgG as described above in Example 2.

### Re-Characterization of Catalytic Antibodies

IgG antibodies are re-characterized as described above in Example 2, except the binding assay used is specific for VEGFr2.

Biological Studies

Animal Models

Anti-angiogenesis cancer therapies are primarily useful in solid tumors (where tumor mass vascularization is crucial). For this reason, cell culture models are less relevant than animal models. Various mouse models have been used to test the therapeutic efficacy of anti-VEGF antibodies. A well-established model uses human rhabdomyosarcoma cell line A673 (ATCC Manassas, Va.). Cells are injected (10⁶ cells, intraperitoneally) into female Beige nude/xid mice (6-10 weeks old, Charles River, Wilmington, Del.). Antibody and antibiotic are administered to the mice at various dose and time regimens. The regimens depend on a number of predetermined factors including the pharmacokinetics (blood level vs. time profile) and antibiotic and antibody tolerance. At the end of the experiment, mice are sacrificed and tumor size is determined by multiplying width times length.

An alternative model involves human colorectal carcinoma cell lines. The VEGF-dependent human tumor cell lines (LS 174T and Colo 320/205/201) are commercially available (ATCC, Manassas, Va.). Cells are injected subcutaneously (5×10⁶ cells) into pathogen-free Balb/c NCR/NU athymic mice (3-4 weeks old, Simonsen Laboratories, Gilroy, Calif.). Anti-VEGFr2 antibody and antibiotic are administered to the mice at various doses and times. At the end of the study, mice are sacrificed and tumor size is determined by multiplying width times length.

### Example 4

### IL-4

Experimental Details

IL-4 is available in B&T Cell Growth Supplement (B&T CGS; IGEN), a reagent that is normally used as a cell culture additive. B&T CGS is rich in IL-4 (10,000 units/mL). This sterilized source may be used, with or without any purification. Alternatively, a human IL-4-expressing E. coli cell line (ATCC 57592) may be used. IL-4 is purified by standard protein purification methods.

Phage Display

Phage display is carried out as described in Example 2, with the obvious difference in the immunoassay used and source of the target molecule.

HTS

High throughput screening is carried out essentially as described above in Example 2, except that the assay is an immunoassay is specific for IL-4. An ECL-based sandwich immunoassay for IL-4 exists and is available at IGEN.

Directed Evolution

Directed evolution to discover anti-IL-4 catalytic antibodies is carried out as described above in Example 2, except that the target molecule is not be co-expressed with antibodies in E. coli. Instead, IL-4 from IGEN's B&T CGS cell culture medium is added externally to the E. coli culture medium.

Characterization of Discovered Catalytic Antibodies

Discovered antibodies are characterized as described above in Example 2, except the immunoassay is specific for IL-4.

Re-Engineering Antibodies from scFv Fragments to Whole IgG

The scFv fragments are converted to whole IgG as described above in Example 2.

Re-Characterization of Antibodies

IgG antibodies are re-characterized as described above in Example 2, except the immunoassay used is specific for IL-4.

Biological Studies

Cell Culture Model

The simplest and least expensive test of IL-4 inactivating antibodies is a cell culture model. Such a model has been published (Beckmann et al., 1990). The authors tested the effectiveness of non-catalytic antibodies in inhibiting IL-4-induced cell proliferation in culture. The described T-cell line CTLL-2, is commercially available (ATCC, Manassas, Va.).

Animal Models

We may additionally (or alternatively) use animal models. Very good mouse models exist, some of which have been used to test anti-IL-4 biotherapies. Active IL-4 is absolutely required for the generation of IgE responses. Injection of anti-IgD into mice results in a large increase in IgE production. Administration of the IL-4 soluble receptor blocked this increase (Maliszewski et al., 1994). A second established mouse model involves aerosolized antigen inhalation (ovalbumin), resulting in an elevation of IL-4 and IgE concentrations. This asthma model has been used to show the effectiveness of soluble IL-4 therapy (Henderson et al., 2000). For either animal model, the treatment will consist of injection of both antibody and antibiotic (either Cefoxitin or Cefotaxime). We will use various dosing and timing regimens for antibody and antibiotic. Regimens will depend on a number of pre-determined factors including the pharmacokinetics (blood level vs. time profile) and antibiotic and antibody tolerance.

### Example 5

### IL-6

Experimental Details

IL-6 is obtained from IGEN's line of commercially available cell culture products, Hybridoma Growth Factor (HCF) Supplement, normally used as a cell culture additive. HCF is rich in IL-6. This sterilized source may be used, with or without further purification. Alternatively, IL-6 may be isolated from an IL-6 overproducing cell line, available from the ATCC (Manassas, Va.).

Phage Display

Phage display is carried out as described above in Example 2, except that IL-6 is used as the target molecule.

HTS

High throughput screening is carried out essentially as described above in Example 2, except that the immunoassay is specific for IL-6. An ECL-based sandwich immunoassay for IL-6 has been developed at IGEN and is available for use.

Directed Evolution

Directed evolution to discover anti-IL-6 antibodies is performed as described in Example 2, except that the target molecule is not be co-expressed with antibodies in E. coli. Instead, IL-6 from IGEN's HCF supplement is added to the E. coli culture medium.

Characterization of Discovered Catalytic Antibodies

Discovered abzymes are characterized as described above in Example 2, except the immunoassay is specific for IL-6

Re-Engineering Antibodies from scFv Fragments to Whole IgG

The scFv fragments are converted to whole IgG by the methods outlined in Example 2.

Re-Characterization of Catalytic Antibodies

IgG antibodies are re-characterized as described above in Example 2, except the immunoassay used is specific for IL-6.

Biological Studies

Rheumatoid Arthritis (RA)

Catalytic antibodies directed to IL-6 are tested as described above in Example 2 in the animal model designed for RA.

Multiple Myeloma (MM)

Two well-established systems are used to test the biological effects of anti-IL-6 abzymes on myeloma cells. One test is a simple proliferation assay using hybridoma cells (fusions of mouse spleen cells and mouse myeloma cells). The mouse myeloma cells used are Sp2/0 cells. Briefly, the hybridoma cells are cultured in IL-6-containing media along with anti-IL-6 antibody and antibiotic. After 2-4 days, cell proliferation is quantitated by measuring [³ H]-thymidine uptake. A number of IL-6-requiring hybridoma cell lines are available from ATCC (Sp2/mIL-6, SA22, R2-9A5. The second model uses human MM cell line, U266 (ATCC, Manassas, Va.). U266 cells were used to test the biological effects of anti-IL-6 receptor antibody.

The above description of the invention is intended to be illustrative and not limiting. Various changes or modifications in the embodiments described may occur to those skilled in the art. These can be made without departing from the spirit or scope of the invention.

### REFERENCES

1. ACS; American Cancer Society web site: www.cancer.org.

2. Angeles, T. S. et al. (1993), Biochemistry 32, 12128.

3. Anne, S. & Reisman, R. E. (1995), Ann. Allergy Asthma Immunol. 74, 167.

4. Arkin, M. R. & Wells, J. A (1998), J. Mol. Biol. 284, 1083.

5. Arnold, F. H. (1998), Acc. Chem. Res. 31, 125.

6. Bataille, R. (1997), Seminars in Hematology 34, 23.

7. Bazzoni, F. & Butler, B. (1996), New Engl. J. Med. 334, 1717.

8. Beatty, J. (1992), Cancer Supplement 70, 1425.

9. Beckmann, M. P. et al. (1990), J. Immunol. 144, 4212.

10. Black, M. E. & Loeb, L. A. (1993), Biochemistry 32, 11618.

11. Blackburn, G. F. et al. (1991), Clin. Chem. 37, 1534.

12. Bolger, R. (1999) Drug Discov. Today 4, 251.

13. Borish, L. C. (1999), Am. J. Respir. Crit. Care Med. 160, 1816.

14. Brakenhoff et al. (1996), FEBS Lett. 395, 235.

15. Carballo, E. et al. (1998), Science 281, 1001.

16. Chirinos-Rohas, C. L. (1998), J. Immunol. 161, 5621.

17. Danheiser, S. (1999), “Outlook for Cytokine and Anticytokine Therapeutics in Hematology, Inflammatory and Autoimmune Disorders, Infectious Disease, and Cancer,” Decision Resources Inc, Waltham, Mass.

18. Datamonitor (1999), “Market Dynamics 1999 Asthma: Seven Country Analysis and Perspective”, Datamonitor Inc.: New York.

19. Demartis, S. et al. (1999), J. Mol. Biol. 286, 617.

20. DeVita, V. et al. (1997), “Cancer: Principles & Practice of Oncology,” (Lippincott-Raven)

21. Domingues, H. et al. (1999), Nature Struct. Biol. 6, 652.

22. Ehlers, M. et al. (1996), J. Interferon Cytokine Res. 16, 569.

23. Eigler, A. et al. (1997), Immunol. Today 18, 487.

24. Elliot, M. J. et al. (1994), Lancet 344, 1125.

25. Ferrara, N. (1999), Kidney Int. 56, 794.

26. Fujii, I. et al. (1998), Nature Biotechnol. 16, 463.

27. Goldinger, M. (1999), “Colorectal Cancer: An Overview.” http://home.swipnet.se/crc/crc.htm

28. Grayling, R. (1998), “IBC Conference Proceedings: Directed Evolution of Industrial Enzymes”, IBC USA Conferences Inc: Westborough, Mass.

29. Hage, T. et al. (1999), Cell 97, 271.

30. Henderson, W. R. et al. J. Immunol. (2000) 164, 1086.

31. Hirano, T. (1998), Intern. Rev. Immunol. 16, 249.

32. Huang, Y. W. & Vitetta, E. S. (1993), Hybridoma 12, 621.

33. Huls, G. A. et al. (1999), Nature Biotechnol. 17, 276.

34. Huston, J. S. (1993), Cell. Biophys. 22, 189.

35. Isomaki, P. & Punnonen, J. (1997), Ann. Med. 29, 499.

36. Jacobs, J. W. (1991), Bio/Technology 9, 258.

37. Jones, E. Y. et al. (1989), Nature 338, 225.

38. Joyce, G. F. (1994), Curr. Opin. Struct. Biol. 4, 331.

39. Kalai, M. et al. (1997), Blood 89, 1319.

40. Kahne, D. & Still, C. J. (1998), Am. Chem. Soc. 110, 7529.

41. Klagsbrun, M. & D'Amore, P.A.(1996), Cytokine Growth Factor Rev. 7, 259.

42. Klareskog, L. & McDevitt, H. (1999), Curr. Opin. Immunol. 11, 657.

43. Leonidas, L. L. (1998), www.e-asthma.com

44. Littlehales, C. (1999), Modem Drug Discovery, January/February, 21.

45. Liu, H. et al. (1997), Biochem. Mol. Biol. Int. 42, 1045.

46. Lu, Z. Y. (1995), Blood 86, 3123.

47. Ma, S. et al. (1997), Bioconj. Chem. 8, 695.

48. Magi, B. et al.( 1995), Electrophoresis 16, 1190.

49. Maini, R. N. et al. (1997), APMIS 105, 257.

50. Maliszewski, C. R. et al. (1994), Proc. Soc. Exp. Biol. Med. 206, 233.

51. Mao, S. et al. (1999), Proc. Natl. Acad. Sci. USA 96, 6953.

52. Martin, M. T. (1996), Drug Discovery Today 1, 239.

53. Martiny-Baron, G. & Marme, D. (1995) Curr. Opin. Biotechnol. 6, 675.

54. Massey, R. J. 1992, Biomedical Products, October.

55. Meldrum, D. R. (1998), Am. J. Physiol. 274, R577.

56. Mordenti, J. et al. (1999), Toxicol. Pathol. 27, 14.

57. Nelson, T. (1999), “Redefining Modem Medicine: Therapeutic Proteins and Therapeutic Antibodies,” (Dain Rauscher Wessels)

58. Pescovitz, M. D. (1999), Transplant. Proc. 31, 1201.

59. Pollack, S. J. et al. (1986), Science 234, 1570.

60. Reed, C. et al. (1997), Protein Engin. 10, 1101.

61. Rustgi A. (1995), “Gastrointestinal Cancers, Biology Diagnosis, & Therapy,” (Lippincott-Raven: Philadelphia)

62. Simon, C. et al., eds. (1993), “Antibiotic Therapy in Clinical Practice,” (Schattauer: New York)

63. Simpson, R. J. et al.( 1997), Protein Science. 6, 929.

64. Smiley, J. A. and Benkovic, S. J. (1994), Proc. Natl. Acad. Sci. USA 91: 8319.

65. Smith, R. M. & Hansen, D. E. (1998), J. Am. Chem. Soc. 120, 8910.

66. Smithrud, R. J. & Benkovic, S. J. (1997), Curr. Opin. Biotechnol. 8, 459.

67. Somers, W. et al. (1997), EMBO J. 16, 989.

68. Sumikawa, H. & Suzuki, E. (1998), Chem. Pharm. Bull. 46, 136.

69. Tang, Y. et al. (1991), Proc. Natl. Acad. Sci. USA 88, 8784.

70. Tracy, K. J. & Cerami, A. (1994), Ann. Rev. Med. 45, 491.

71. Tramontano, A. et al. (1986), Science 234, 1566.

72. Warren, R. S. et al. (1995), J. Clin. Invest. 95, 1789.

73. Wilson, D. R. & Finlay, R. B. (1998), Can. J. Microbiol. 44, 313.

74. Winter, G. et al. (1994), Ann. Rev. Immunol. 12, 433.

75. Witte, L. et al. (1998), Cancer Metastasis Rev. 17, 155.

76. WoodMackenzie (1999), “Anti-Cancers, ” www.woodmackenzie.com/phview/phquant/products/cancer.htm

77. Means, G. E, & Feeney, R. E. “Chemical Modification of Proteins”, Holden Press, San Francisco, 1971.

1. A method of modifying a biologically active target molecule comprising contacting said target molecule with a catalyst capable of chemically modifying said target molecule, said contacting being effected under conditions sufficient for said catalyst to modify said target molecule.

2. The method of claim 1 wherein said target molecule is selected from the group consisting of a protein, peptide, nucleic acid, carbohydrate, cell, subcellular particle, prion, virus, steroid, lipid, receptor, ligand, hormone, gene, enzyme, and cytokine.

3. The method of claim 1 wherein said target molecule is a protein associated with a disease condition.

4. The method of claim 1 wherein the catalyst is an enzyme or a catalytic antibody.

5. The method of claim 4 wherein the catalyst is a catalytic antibody.

6. The method of claim 1 wherein said modification is selected from the group consisting of (a) introducing a chemical moiety to said target molecule; (b) linking two or more target molecules; (c) modulating an activity of said target molecule; (d) deactivating said target molecule; and (e) targeting said target molecule for degradation or clearance.

7. The method of claim 1 wherein said catalyst modifies said target molecule by a method selected from the group consisting of acylation, glycosylation, esterification, and transamidation.

8. The method of claim 7 wherein said catalyst modifies said target molecule by acylation with at least one β-lactam antibiotic.

9. The method of claim 8 wherein said antibiotic is selected from the group consisting of cefoxitin and cefotaxime.

10. The method of claim 2 wherein said target molecule is selected from the group consisting of TNFα, IL-4, IL-6, and VEGFr2.

11. The method of claim 4 wherein said catalyst is a catalytic antibody isolated from a library of antibodies or fragments thereof by one or more methods selected from the group consisting of phage display, in vivo selection, and high throughput screening.

12. The method of claim 11 wherein said library is generated by immunizing an animal with a hapten resembling a combining site of said target molecule, alone or in combination with an agent used to chemically modify said target molecule at said combining site.

13. The method of claim 11 wherein in vivo selection comprises:
(a) subjecting a bacteria in a growth medium to conditions sufficient for said bacteria to express and secrete putative antibodies;
(b) adding said target molecule to said growth medium and/or subjecting said bacteria to conditions sufficient for said bacteria to co-express and secrete said target molecule with said putative antibodies;
(c) adding a toxic concentration of at least one β-lactam antibiotic to said growth medium;
(d) identifying one or more bacterial colonies that survived step (c); and
(e) isolating a catalytic antibody from said colonies identified in step (d).

14. The method of claim 13 wherein said putative antibodies are pre-selected by phage display for putative antibodies having an affinity for an antibiotic-target molecule adduct.

15. The method of claim 13 wherein said one or more β-lactam antibiotics are cefoxitin and cefotaxime, and the toxic concentrations of said antibiotics are 30 μM-50 μM cefoxitin and 0.20 μM-0.60 μM cefotaxime.

16. The method of claim 13 wherein said catalytic antibody catalyzes at least 220-555 turnovers with cefoxitin or 11.2-33.6 turnovers with cefotaxime.

17. A catalytic antibody capable of chemically modifying a biologically active target molecule.

18. The catalytic antibody of claim 17 wherein said target molecule is selected from the group consisting of a protein, peptide, nucleic acid, cell, subcellular particle, prion, virus, steroid, lipid, receptor, ligand, hormone, gene, enzyme, and cytokine.

19. The method of claim 18 wherein said target molecule is a protein associated with a disease condition.

20. The catalytic antibody of claim 17 wherein said catalytic antibody is isolated from a library of antibodies or fragments thereof by one or more methods selected from the group consisting of phage display, in vivo selection, and electrochemiluminescence-based high throughput screening.

21. The catalytic antibody of claim 20 wherein said library is generated by immunizing an animal with a hapten resembling a combining site of said target molecule, alone or in combination with an agent used to chemically modify said target molecule at said combining site.

22. The catalytic antibody of claim 21 wherein in vivo selection comprises:
(a) subjecting a bacteria in a growth medium to conditions sufficient for said bacteria to express and secrete putative antibodies;
(b) adding said target molecule to said growth medium and/or subjecting said bacteria to conditions sufficient for said bacteria to co-express and secrete said target molecule with said putative antibodies;
(c) adding a toxic concentration of at least one β-lactam antibiotic to said growth medium;
(d) identifying one or more bacterial colonies that survived step (c); and
(e) isolating a catalytic antibody from said colonies identified in step (d).

23. The catalytic antibody of claim 22 wherein said putative antibodies are pre-selected by phage display for putative antibodies having an affinity for an antibiotic-target molecule adduct.

24. The catalytic antibody of claim 22 wherein said one or more β-lactam antibiotics are cefoxitin and cefotaxime, and the toxic concentrations of said antibiotics are 20 μM-50 μM cefoxitin and 0.20 μM-0.60 μM cefotaxime.

25. The catalytic antibody of claim 22 wherein said catalytic antibody catalyzes at least 220-555 turnovers with cefoxitin or 11.2-33.6 turnovers with cefotaxime.

26. The catalytic antibody of claim 18 wherein said catalytic antibody chemically modifies and thereby deactivates a target molecule selected from the group consisting of TNFα, IL-4, IL-6, and VEGFr2.

27. A composition comprising a catalyst capable of chemically modifying a biologically active target molecule and a pharmaceutically acceptable carrier or diluent.

28. The composition of claim 27 wherein said target molecule is selected from the group consisting of a protein, peptide, nucleic acid, cell, subcellular particle, prion, virus, steroid, lipid, receptor, ligand, hormone, gene, enzyme, and cytokine.

29. The composition of claim 27 wherein said target molecule is a protein associated with a disease condition.

30. The composition of claim 27 wherein the catalyst is an enzyme or a catalytic antibody.

31. The composition of claim 30 wherein the catalyst is a catalytic antibody.

32. The composition of claim 27 wherein said modification is selected from the group consisting of (a) introducing a chemical moiety to said target molecule; (b) linking two or more target molecules; (c) modulating an activity of said target molecule; (d) deactivating said target molecule; and (e) targeting said target molecule for degradation or clearance.

33. The method of claim 27 wherein said catalyst modifies said target molecule by a method selected from the group consisting of acylation, glycosylation, esterification, and transamidation.

34. A method of treating a disease condition associated with TNFα in a patient in need of said treatment comprising administering to said patient an amount of a catalytic antibody effective to chemically modify and thereby deactivate TNFα.

35. The method of claim 34 wherein said disease condition is selected from the group consisting of rheumatoid arthritis, Crohn's disease, inflammation, septic shock, cachexia, cancer, parasitic infections, allograft rejections, and heart disease.

36. A method of treating a disease condition associated with VEGF in a patient in need of said treatment comprising administering to said patient an amount of a catalytic antibody effective to chemically modify and thereby deactivate VEGF.

37. The method of claim 36 wherein said disease condition is selected from the group consisting of rheumatoid arthritis, colorectal cancer, and metastatic renal cell cancer.

38. A method of treating a disease condition associated with IL-4 in a patient in need of said treatment comprising administering to said patient an amount of a catalytic antibody effective to chemically modify and thereby deactivate IL-4.

39. The method of claim 38 wherein said disease condition is an allergic inflammation associated with allergic asthma, rhinitis, conjunctivitis, and dermatitis.

40. A method of treating a disease condition associated with IL-6 in a patient in need of said treatment comprising administering to said patient an amount of a catalytic antibody effective to chemically modify and thereby deactivate IL-6.

41. The method of claim 40 wherein said disease condition is selected from the group consisting of inflammation, multiple myeloma, renal cell carcinoma, Kaposi's sarcoma, rheumatoid arthritis, Castleman's disease, and acquired immunodeficiency syndrome.

42. A method of modifying a biologically active target molecule comprising contacting said target molecule with a catalyst and a label, wherein said catalyst chemically modifies said target molecule by attaching said label.

43. The method of claim 42 wherein said label is a detectable label.

44. The method of claim 42, wherein the attachment of said label disrupts the biological activity of said target molecule.

45. The method of claim 42, wherein said label is a beta-lactam antibiotic and said catalyst catalyzes the acylation of said target molecule by said beta-lactam.
